(19) United States
(12) Patent Application Publication
(10) Pub. No.: US 2026/0022168 A1
(43) Pub. Date: Jan. 22, 2026

(54) METHOD OF CONCENTRATING A SOLUTION OR FORMULATION

(71) Applicant: Kodiak Sciences Inc., Palo Alto, CA (US)

(72) Inventors: Wah Yuen To, San Mateo, CA (US); Martin Nussbaumer, Wabern (CH); Dorian Schläfli, Venthône (CH); Laurent Ducry, Sierre (CH)

(21) Appl. No.: 19/114,405

(22) PCT Filed: Sep. 21, 2023
(86) PCT No.: PCT/US2023/074805

§ 371 Date: Mar. 21, 2025

Related U.S. Application Data
(60) Provisional application No. 63/376,917, filed on Sep. 23, 2022.

(51) Int. Cl. C07K 16/24; C07K 1/14; C07K 16/22

(52) U.S. Cl. CPC C07K 16/248; C07K 1/14; C07K 16/22; C07K 2317/31; C07K 2317/51; C07K 2317/515; C07K 2317/565; C07K 2317/76; C07K 2319/00

(57) ABSTRACT

Some aspects of the present disclosure are directed towards the concentration or reduction of a solution, formulation, mixture, or compound comprising a protein conjugated to a polymer. In some embodiments, the method can involve applying a vacuum concentration process to the antibody conjugate comprising solution.

INCORPORATION BY REFERENCE TO ANY PRIORITY APPLICATIONS

This application claims the benefit of U.S. Provisional Ser. No. 63/376,917, filed Sep. 23, 2022 which is hereby incorporated by reference in its entirety.

### REFERENCE TO SEQUENCE LISTING

The present application is being filed along with a Sequence Listing in electronic format. The Sequence Listing is provided as a file entitled KDIAK099WO_SEQ_LIST.xml, created Sep. 21, 2023, which is 73,813 bytes in size. The information in the electronic format of the Sequence Listing is incorporated herein by reference in its entirety.

### BACKGROUND

### Field

The present disclosure is generally related to methods of concentrating a solution or formulation. Specifically, some aspects of the present disclosure are directed to a method of concentrating a viscous protein solution or therapeutic formulation. Some embodiments are directed towards methods of reducing the volume of a solution or therapeutic formulation. Current methods of delivering antibody based therapeutics, including by intravenous infusion or subcutaneous infusion, require highly concentrated therapeutic formulations which may result in highly viscous solutions. High viscosities may pose difficulties during bioprocessing, downstream filtration, or diafiltration. High viscosities may also lead to increased processing times, increased drug destabilization, and increased manufacturing costs. Thus, improved methods of concentrating viscous solutions are needed.

### SUMMARY

Provided herein are methods of concentrating a viscous protein solution or formulation. Also provided herein are methods of reducing the volume of a therapeutic formulation. In some embodiments, a method of concentrating a viscous protein solution is disclosed, the method comprising: providing a first solution comprising protein, and reducing a volume of the first solution via evaporative concentration, wherein a vacuum is applied during evaporative concentration of at least below 100 mbar, to thereby produce a second solution, wherein the second solution has a viscosity between 400 to 4000 mPa.s or cP.

In some embodiments, a method of reducing a volume of a therapeutic formulation is disclosed. The method comprising: providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution; wherein the second solution has a viscosity between 400 to 4000 mPa.s or cP.

In some embodiments, a method of reducing a volume of a therapeutic formulation is disclosed. The method comprising: providing a first solution, the first solution comprising at least 0.001% polysorbate and a conjugate, wherein the conjugate comprises a polymer conjugated to an antibody or a small-molecule drug or both, wherein the first solution is at a first volume; and applying a vacuum of at least below 100 mbar to the first solution to reduce the first volume to a second volume by evaporative concentration to thereby produce a second solution, wherein the second solution has a viscosity between 400 to 4000 mPa.s or cP, wherein evaporative concentration is carried out at less than 40° C., and wherein the vacuum of at least below 100 mbar is established over at least 15 minutes to reduce foaming

In some embodiments, a method of concentrating a viscous protein solution is disclosed, the method comprising: providing a first solution comprising protein; and reducing a volume of the first solution via evaporative concentration, wherein a vacuum is applied during evaporative concentration of at least below 100 mbar, to thereby produce a second solution, wherein the second solution has a viscosity between 250 to 4000 mPa.s or cP.

In some embodiments, a method of reducing a volume of a therapeutic formulation is disclosed. The method comprising: providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution, wherein the second solution has a viscosity between 250 to 4000 mPa.s or cP.

In some embodiments, a method of reducing a volume of a therapeutic formulation is disclosed. The method comprising: providing a first solution, the first solution comprising at least 0.001% polysorbate and a conjugate, wherein the conjugate comprises a polymer conjugated to an antibody or a small-molecule drug or both, wherein the first solution is at a first volume; and applying a vacuum of at least below 100 mbar to the first solution to reduce the first volume to a second volume by evaporative concentration to thereby produce a second solution, wherein the second solution has a viscosity between 250 to 4000 mPa.s or cP, wherein evaporative concentration is carried out at less than 40° C., and wherein the vacuum of at least below 100 mbar is established over at least 15 minutes to reduce foaming.

In some embodiments, the disclosed methods further comprise agitation of the first and/or second solution. In some embodiments, the first and/or second solution are agitated via stirring. In some embodiments, the solution is stirred for a period of time lasting at least about 15 minutes. In some embodiments, the solution is stirred for a period of time lasting between about 30 minutes and 6 hours. In some embodiments, the solution is stirred for a period of time lasting at between about 24 and 96 hours. In some embodiments, the solution is stirred for a period of time lasting at between about 48 and 72 hours. In some embodiments, the solution is stirred for a period of time lasting about 48 hours. In some embodiments, the solution is stirred for a period of time lasting about 60 hours. In some embodiments, the solution is stirred for a period of time lasting about 72 hours. In some embodiments, the solution is stirred at up to about 100 RPM. In some embodiments, the solution is stirred at up to about 25 RPM. In some embodiments, the solution is stirred at up to about 15 RPM. In some embodiments, the solution is stirred continuously. In some embodiments, the solution is stirred intermittently. In some embodiments, the solution is stirred to homogeneity.

In some embodiments, the first solution reduced by evaporative concentration is reduced by between about 20% and 80%. In some embodiments, the first solution reduced by evaporative concentration is reduced by between about 30% and 80%. In some embodiments, the first solution reduced by evaporative concentration is reduced by between about 40% and 70%. In some embodiments, the first solution reduced by evaporative concentration is reduced by up to about 60%. In some embodiments, the first solution reduced by evaporative concentration is reduced by up to about 2.5-fold.

In some embodiments, the evaporative concentration is carried out at vacuum below at least 100 mbar. In some embodiments, the evaporative concentration is carried out at vacuum below at least 50 mbar. In some embodiments, the vacuum applied is at least below 30 mbar. In some embodiments, the evaporative concentration is carried out at vacuum of about 17 mbar. In some embodiments, the vacuum below 100 mbar is lowered over a period of up to about 30 minutes. In some embodiments, the pressure is reduced from atmospheric pressure to about 17 mbar at a rate of about 90 mbar/min. In some embodiments, the pressure is reduced from below about 100 mbar to the target pressure at a rate of about 1 mbar/min. In some embodiments, the vacuum can be adjusted if the temperature is adjusted accordingly.

In some embodiments, evaporative concentration is carried out at a vacuum pressure of 10 mbar and a temperature of 7° C. In some embodiments, evaporative concentration is carried out at a vacuum pressure of 17 mbar and a temperature of 15° C. In some embodiments, evaporative concentration is carried out at a vacuum pressure of 24 mbar and a temperature of 21° C. In some embodiments, evaporative concentration is carried out at a vacuum pressure of 30 mbar and a temperature of 24° C. In some embodiments, evaporative concentration is carried out at a vacuum pressure of 42 mbar and a temperature of 30° C. In some embodiments, evaporative concentration is carried out at a vacuum pressure of 73 mbar is used and a temperature of 40° C. In some embodiments, a vacuum pressure of 123 mbar is used at a temperature of 50° C.

In some embodiments, the vacuum to the target pressure is increased over a period of time lasting at least about 15 minutes. In some embodiments, the vacuum to the target pressure is increased over a period of time lasting between about 30 minutes and 6 hours. In some embodiments, the vacuum to the target pressure is increased over a period of time lasting between about 45 minutes and 3 hours. In some embodiments, the vacuum to the target pressure is increased over a period of time lasting at least about 1.5 hours. An increase in vacuum in this context denotes that the pressure is decreased, thereby increasing the vacuum.

In some embodiments, the vacuum applied to the first solution is for up to about 96 hours. In some embodiments, the vacuum applied to the first solution is for between about 48 and 72 hours. In some embodiments, the vacuum applied to the first solution is for about 48 hours. In some embodiments, the vacuum applied to the first solution is for about 60 hours. In some embodiments, the vacuum applied to the first solution is for about 72 hours.

In some embodiments, the evaporative concentration process is monitored on-line. In some embodiments, the concentration measurement comprises using ultraviolet light. In some embodiments, the concentration measurement is made using FlowVPE. In some embodiments, the volume measurement comprises using one or more of: radar, conductivity, viscosity, or osmolality.

In some embodiments, the volume of the first solution is between about 25 L and 100 L.

In some embodiments, the first solution further comprises a surfactant.

In some embodiments, the first solution comprises between about 0.001% to 0.2% surfactant. In some embodiments, the first solution comprises about 0.01% surfactant. In some embodiments, the surfactant comprises fatty acid esters, sorbitol, and/or sorbitol derivatives. In some embodiments, the surfactant comprises polysorbate. In some embodiments, the polysorbate comprises Polysorbate-20. In some embodiments, the polysorbate comprises Polysorbate-80. In some embodiments, the first solution comprises polysorbate; and wherein said first solution is concentrated such that the concentration of polysorbate in the second solution is between 1-fold and 5-fold higher.

In some embodiments, the second solution has a viscosity of 750 to 2000 mPa.s or cP. In some embodiments, the second solution has a viscosity of 250 to 2000 mPa.s or cP. In some embodiments, the second solution has a viscosity of about 1000 mPa.s or cP. In some embodiments, the second solution has a viscosity of about 315 mPa.s or cP.

In some embodiments, evaporative concentration is carried out at a temperature between about 40 degrees C. and freezing. In some embodiments, evaporative concentration is carried out at a temperature between about 25 degrees C. and freezing. In some embodiments, evaporative concentration is carried out at temperature below about 15 degrees C. In some embodiments, evaporative concentration is carried out at a temperature between about 5 degrees C. and freezing.

In some embodiments, the concentration of the protein in the second solution is between 40 mg/mL and 60 mg/mL. In some embodiments, the concentration of the protein in the second solution is between 45.0 mg/mL and 52.5 mg/mL. In some embodiments, the concentration of the protein in the second solution is about 47.5 mg/mL.

In some embodiments, the disclosed methods further comprise venting the second volume. In some embodiments, the disclosed methods further comprise filtering the concentrated viscous protein solution. In some embodiments, the disclosed methods further comprise collecting quality control samples during the evaporative concentration process. In some embodiments, the disclosed methods further comprise storage of the concentrated viscous protein solution.

In some embodiments, the therapeutic formulation is a pharmaceutical composition comprising an isolated antagonistic antibody, and a pharmaceutically acceptable carrier. In some embodiments, the therapeutic formulation comprises a formulation for the treatment or prophylaxis of an ocular disorder. In some embodiments, the antibody is a conjugated antibody.

In some embodiments, the conjugated antibody is an isolated antagonist antibody comprising: a CDRH1 that is a CDRH1 in SEQ ID NO: 28; a CDRH1 that is a CDRH1 in SEQ ID NO: 29; a CDRH1 that is a CDRH1 in SEQ ID NO: 30; a CDRH1 that is a CDRH1 in SEQ ID NO: 33; a CDRH1 that is a CDRH1 in SEQ ID NO: 34; a CDRH1 that is a CDRH1 in SEQ ID NO: 35; at least one of the following mutations (EU numbering): L234A, L235A, and G237A; and at least one of the following mutations (EU numbering): Q347C or L443C.

In some embodiments, the conjugated antibody comprises an isolated antagonist IL-6 antibody comprising: a heavy chain amino acid variable region that comprises a heavy chain that has a sequence of at least one of SEQ ID NOs. 2-17, 21, 22, 36-42; and a light chain amino acid variable region that comprises the light chain that has a sequence of at least one of SEQ ID NOs. 18-20, 23-25.

In some embodiments, the conjugated antibody comprises an anti-VEGF antibody. In some embodiments, the anti-VEGF antibody heavy chain comprises a CDRH1that is a CDRH1 in SEQ ID NO.: 51; a CDRH2 that is a CDRH2 in SEQ ID NO.: 52; and a CDRH3 that is a CDRH3 in SEQ ID NO: 53. In some embodiments, the anti-VEGF antibody heavy chain comprises a CDRL1 that is a CDRL1 in SEQ ID NO.: 54; a CDRL2 that is a CDRL2 in SEQ ID NO.: 55; and a CDRL3 that is a CDRL3 in SEQ ID NO: 56. In some embodiments, the anti-VEGF antibody comprises: a heavy chain; wherein the heavy chain comprises a CDRH1 that is a CDRH1 in SEQ ID NO.: 51; a CDRH2 that is a CDRH2 in SEQ ID NO.: 52; a CDRH3 that is a CDRH3 in SEQ ID NO: 53; a light chain; wherein the heavy chain comprises a CDRL1 that is a CDRL1 in SEQ ID NO.: 54; a CDRL2 that is a CDRL2 in SEQ ID NO.: 55; a CDRL3 that is a CDRL3 in SEQ ID NO: 56; and wherein the heavy chain constant domain of the anti-VEGF-A antibody has one or more mutations relative to the constant domain of human IgG1 to modulate effector function, wherein the mutations are to one or more of the following amino acid positions (EU numbering): E233X, L234X, L235X, G236X, G237X, A327X, A330X, and P331X wherein X is any natural or unnatural amino acid, wherein the mutations are selected from the group consisting of (EU numbering): E233P, L234V, L234A, L235A, G237A, A327G, A330S, and P331S

In some embodiments, the conjugated antibody comprises an isolated antibody that specifically binds to complement factor D (CFD) and directly inhibits a proteolytic activity of CFD, wherein the antibody comprises: a heavy chain variable region (VH) comprising: a VH complementarity determining region 1 (CDRH1) that is the CDRH1 in SEQ ID NO: 43; a CDRH2 that is the CDRH2 in SEQ ID NO: 43; and a CDRH3 that is the CDRH3 in SEQ ID NO: 43; and a light chain variable region (VL) comprising: a VL complementarity determining region 1 (CDRL1) comprising H31, N33, G34 or E34 or F34 or S34, D35 or E35, T36 or S36 or V36, Y37, L38 or 138, and E39 (EU numbering); a CDRL2 comprising L51 or H51, 153 or V53, and K55 (EU numbering); and a CDRL3 comprising F94or L94, Q95, G96, S97, V99 or N99 or Q99 or W99, P100, and P101 or V101 (EU numbering).

In some embodiments, the conjugated antibody comprises an isolated antagonistic antibody that binds to CFD, the antibody comprising: a VH comprising: a CDRH1 that is the CDRH1 in SEQ ID NO: 43; a CDRH2 that is the CDRH2 in SEQ ID NO: 43; a CDRH3 that is the CDRH3 in SEQ ID NO: 43; a VL comprising: a CDRL1 that is the CDRL1 in SEQ ID NO: 44; a CDRL2 that is the CDRL2 in SEQ ID NO: 44; a CDRL3 that is the CDRL3 in SEQ ID NO: 44; at least one of the following mutations (EU numbering): L234A, L235A, and G237A; and at least one of the following mutations (EU numbering): Q347C or L443C.

In some embodiments, the conjugate has the following structure:

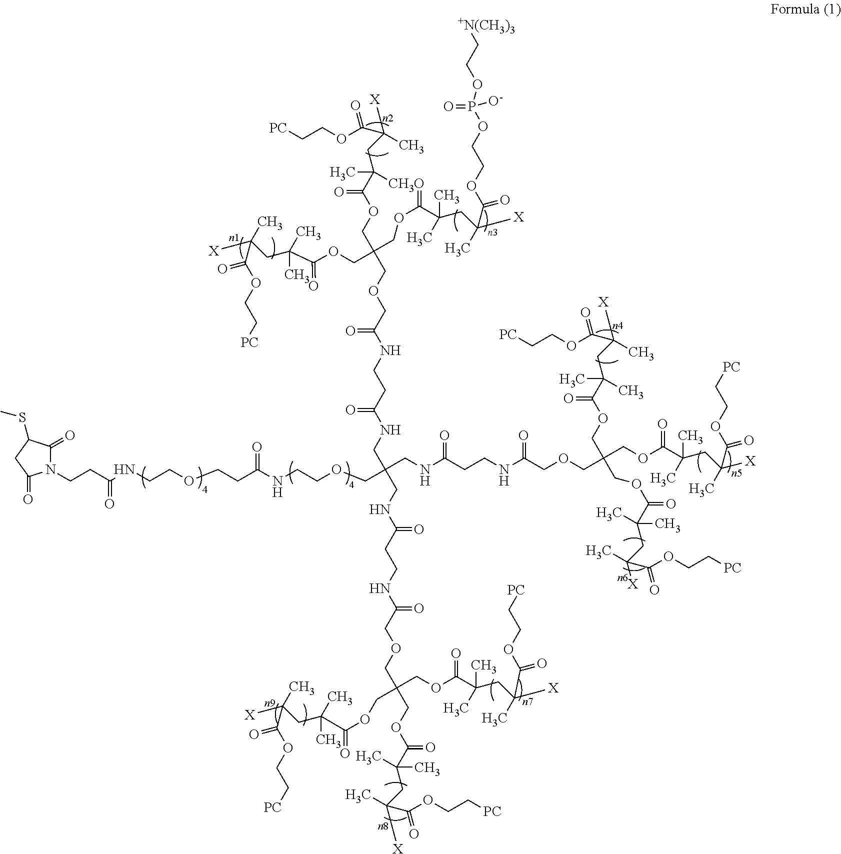

wherein: each heavy chain of the antibody is denoted by the letter H, and each light chain of the antibody is denoted by the letter L; the polymer is bonded to the antibody through the sulfhydryl of C443 (EU numbering), which bond is depicted on one of the heavy chains; PC is

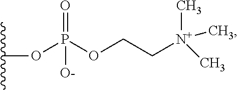 [SMILES: *OP(=O)([O-])OCC[N+](C)(C)C]

where the curvy line indicates the point of attachment to the rest of the polymer, where X=a) OR where R=H, methyl, ethyl, propyl, isopropyl, b) H, or c) any halide, including Br; and n1, n2, n3, n4, n5, n6, n7, n8 and n9 are the same or different such that the sum of n1, n2, n3, n4, n5, n6, n6, n7, n8 and n9 is 2500 plus or minus 15%.

In some embodiments, the protein comprises a fusion protein. In some embodiments, the protein is a fusion protein comprising a vascular endothelial growth factor (hereinafter “VGEF”) antagonist linked to a platelet-derived growth factor (herein after “PDGFR”) extracellular trap segment, wherein the PDGFR extracellular trap segment comprises domains D1-D3 of PDGFR-β.

In some embodiments, the antibody is conjugated to a polymer. In some embodiments, 60 to 90% of the antibody is conjugated to a polymer. In some embodiments, the fusion protein is conjugated to a polymer. In some embodiments, the polymer is covalently bonded to the antibody at a cysteine outside a variable region of the antibody wherein said cysteine has been added via recombinant technology. In some embodiments, the polymer is a biopolymer. In some embodiments, the biopolymer comprises an aflibercept biopolymer. In some embodiments, the polymer comprises a phosphorylcholine polymer. In some embodiments, the phosphorylcholine containing polymer comprises 2-(methacryloyloxyethyl)-2′-(trimethylammonium) ethyl phosphate (MPC) monomers as set forth below:

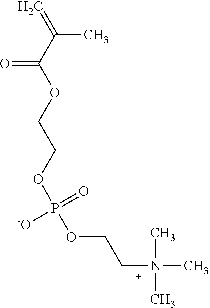

In some embodiments, the polymer has three or more arms or is synthesized with an initiator comprising 3 or more polymer initiation sites. In some embodiments, the polymer has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites. In some embodiments, the polymer has 2, 3, 6, or 9 arms or is synthesized with an initiator comprising 2, 3, 6, or 9 polymer initiation sites. In some embodiments, the polymer has 9 arms or is synthesized with an initiator comprising 9 polymer initiation sites.

In some embodiments, the polymer has a molecular weight between about 300,000 and about 1,750,000 Da as measured by size exclusion chromatography-multi angle light scattering. In some embodiments, the polymer has a molecular weight between about 500,000 and about 1,000,000 Da. In some embodiments, the polymer has a molecular weight between about 750,000 to about 850,000 Da.

In some embodiments, the polymer comprises a zwitterionic monomer, wherein the zwitterionic monomer is selected from the group consisting of HEMA-phosphorylcholine, PEG, biocompatible fatty acids and derivatives thereof, Hydroxy Alkyl Starch (HAS), Hydroxy Ethyl Starch (HES), Poly Ethylene Glycol (PEG), Poly (Glyx-Sery) (HAP), Hyaluronic Acid (HA), Heparosan polymers (HEP), Fleximers, Dextran, Poly-sialic acids (PSA), FC domains, Transferrin, 25 Albumin, Elastin Like Peptides (ELP), CTP peptides, and FCRn binding peptides.

### BRIEF DESCRIPTION OF THE DRAWINGS

FIG. 1 shows a flowchart illustrating some embodiments of a method for concentrating a viscous protein solution.

FIG. 2 shows a flowchart illustrating some embodiments of a method for reducing the volume of a therapeutic formulation.

FIG. 3 shows a flowchart illustrating some embodiments of a method for reducing the volume of a therapeutic formulation.

FIG. 4 shows some embodiments of a polynucleotide sequence of a mature form of human complement factor D polynucleotide. (SEQ ID NO: 1)

FIG. 5 shows some embodiments of a polynucleotide sequence of a mAb KCD005 heavy chain polynucleotide. (SEQ ID NO: 2)

FIG. 6 shows some embodiments of a polynucleotide sequence of a mAb KCD005 light chain polynucleotide. (SEQ ID NO: 3)

FIG. 7 shows some embodiments of a polynucleotide sequence of a CDRH1 polynucleotide. (SEQ ID NO: 4)

FIG. 8 shows some embodiments of a polynucleotide sequence of a CDRH2 polynucleotide. (SEQ ID NO: 5)

FIG. 9 shows some embodiments of a polynucleotide sequence of a CDRH3 polynucleotide. (SEQ ID NO: 6)

FIG. 10 shows some embodiments of a polynucleotide sequence of a CDRL1 polynucleotide. (SEQ ID NO: 7)

FIG. 11 shows some embodiments a polynucleotide sequence of a CDRL2 polynucleotide. (SEQ ID NO: 8)

FIG. 12 shows some embodiments of a polynucleotide sequence of a mAb KCD023 heavy chain polynucleotide. (SEQ ID NO: 9)

FIG. 13 shows some embodiments of a polynucleotide sequence of a mAb KCD023 light chain polynucleotide. (SEQ ID NO: 10)

FIG. 14 shows some embodiments of a polynucleotide sequence of a mAb KCD036 heavy chain polynucleotide. (SEQ ID NO: 11)

FIG. 15 shows some embodiments of a polynucleotide sequence of a mAb KCD036 light chain polynucleotide. (SEQ ID NO: 12)

FIG. 16 shows some embodiments of a polynucleotide sequence of a mAb KCD040 heavy chain polynucleotide. (SEQ ID NO: 13)

FIG. 17 shows some embodiments of a polynucleotide sequence of a mAb KCD040 light chain polynucleotide. (SEQ ID NO: 14)

FIG. 18 shows some embodiments of a polynucleotide sequence of a mAb KCD042 heavy chain polynucleotide. (SEQ ID NO: 15)

FIG. 19 shows some embodiments of a polynucleotide sequence of a mAb KCD042 light chain polynucleotide. (SEQ ID NO: 16)

FIG. 20 shows some embodiments of a polynucleotide sequence of a mAb KCD044 heavy chain polynucleotide. (SEQ ID NO: 17)

FIG. 21 shows some embodiments of a polynucleotide sequence of a mAb KCD044 light chain polynucleotide. (SEQ ID NO: 18)

FIG. 22 shows some embodiments of a polynucleotide sequence of a mAb KCD047 heavy chain polynucleotide. (SEQ ID NO: 19)

FIG. 23 shows some embodiments of a polynucleotide sequence of a mAb KCD047 light chain polynucleotide. (SEQ ID NO: 20)

FIG. 24 shows some embodiments of a polynucleotide sequence of a KCD005 H3 polynucleotide. (SEQ ID NO: 21)

FIG. 25 shows some embodiments of a polynucleotide sequence of a KCD005 L1 polynucleotide. (SEQ ID NO: 22)

FIG. 26 shows some embodiments of a polynucleotide sequence of a KCD005 L2 polynucleotide. (SEQ ID NO: 23)

FIG. 27 shows some embodiments of a polynucleotide sequence of a KCD005 L3 polynucleotide. (SEQ ID NO: 24)

FIG. 28 shows some embodiments of a polynucleotide sequence of a KCD009 H1 polynucleotide. (SEQ ID NO: 25)

FIG. 29 shows some embodiments of a polynucleotide sequence of a mAb KCD208 light chain polynucleotide. (SEQ ID NO: 26)

FIG. 30 shows some embodiments of a polynucleotide sequence of a mAb KCD214 heavy chain polynucleotide. (SEQ ID NO: 27)

FIG. 31 shows some embodiments of a polynucleotide sequence of a KCD104 H2 polynucleotide. (SEQ ID NO: 28)

FIG. 32 shows some embodiments of a polynucleotide sequence of a KCD104 H3 polynucleotide. (SEQ ID NO: 29)

FIG. 33 shows some embodiments of a polynucleotide sequence of a KCD104 L1 polynucleotide. (SEQ ID NO: 30)

FIG. 34 shows some embodiments of a polynucleotide sequence of a Exemplary human IgG1 heavy chain polynucleotide. (SEQ ID NO: 31)

FIG. 35 shows some embodiments of a polynucleotide sequence of a Exemplary human kappa light chain polynucleotide. (SEQ ID NO: 32)

FIG. 36 shows some embodiments of a polynucleotide sequence of a KCD122 H3 polynucleotide. (SEQ ID NO: 33)

FIG. 37 shows some embodiments of a polynucleotide sequence of a KCD122 L1 polynucleotide. (SEQ ID NO: 34)

FIG. 38 shows some embodiments of a polynucleotide sequence of a KCD122 L2 polynucleotide. (SEQ ID NO: 35)

FIG. 39 shows some embodiments of a polynucleotide sequence of a KCD224 L3 polynucleotide. (SEQ ID NO: 36)

FIG. 40 shows some embodiments of a polynucleotide sequence of a mAb KCD002 heavy chain polynucleotide. (SEQ ID NO: 37)

FIG. 41 shows some embodiments of a polynucleotide sequence of a mAb KCD002 light chain polynucleotide. (SEQ ID NO: 38)

FIG. 42 shows some embodiments of a polynucleotide sequence of a mAb KCD003 heavy chain polynucleotide. (SEQ ID NO: 39)

FIG. 43 shows some embodiments of a polynucleotide sequence of a mAb KCD003 light chain polynucleotide. (SEQ ID NO: 40)

FIG. 44 shows some embodiments of a polynucleotide sequence of a mAb KCD005 heavy chain polynucleotide. (SEQ ID NO: 41)

FIG. 45 shows some embodiments of a polynucleotide sequence of a mAb KCD005 light chain polynucleotide. (SEQ ID NO: 42)

FIG. 46 shows some embodiments of a polynucleotide sequence of a 34I54I59D84S heavy chain polynucleotide. (SEQ ID NO: 43)

FIG. 47 shows some embodiments of a polynucleotide sequence of a 54R101V light chain p polynucleotide. (SEQ ID NO: 44)

FIG. 48 shows some embodiments of a polynucleotide sequence of a HCDR1 polynucleotide. (SEQ ID NO: 45)

FIG. 49 shows some embodiments of a polynucleotide sequence of a HCDR2 polynucleotide. (SEQ ID NO: 46)

FIG. 50 shows some embodiments of a polynucleotide sequence of a HCDR3 polynucleotide. (SEQ ID NO: 47)

FIG. 51 shows some embodiments of a polynucleotide sequence of a LCDR1 polynucleotide. (SEQ ID NO: 48)

FIG. 52 shows some embodiments of a polynucleotide sequence of a LCDR2 polynucleotide. (SEQ ID NO: 49)

FIG. 53 shows some embodiments of a polynucleotide sequence of a LCDR3 polynucleotide. (SEQ ID NO: 50)

FIG. 54 shows some embodiments of a polynucleotide sequence of a HCDR1 polynucleotide. (SEQ ID NO: 51)

FIG. 55 shows some embodiments of a polynucleotide sequence of a HCDR2 polynucleotide. (SEQ ID NO: 52)

FIG. 56 shows some embodiments of a polynucleotide sequence of a HCDR3 polynucleotide. (SEQ ID NO: 53)

FIG. 57 shows some embodiments of a polynucleotide sequence of a LCDR1 polynucleotide. (SEQ ID NO: 54)

FIG. 58 shows some embodiments of a polynucleotide sequence of a LCDR2 polynucleotide. (SEQ ID NO: 55)

FIG. 59 shows some embodiments of a polynucleotide sequence of a LCDR3 polynucleotide. (SEQ ID NO: 56)

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the presently disclosed invention is not limited by any of the particular embodiments described below. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

Some aspects of the present disclosure are directed towards the concentration or reduction of a solution, formulation, mixture, or compound comprising a protein conjugated to a polymer. In some embodiments, the method can involve applying a vacuum concentration process to the antibody conjugate comprising solution.

### Definitions

All terms used herein have their plain and ordinary meaning as understood by those skilled in the art. For example:

The term “concentration” can include the measure of the amount of any one or more sub-components, analytes, ingredients, or constituents of a solution, formulation, or compound. To concentrate a sample can mean to increase the concentration of any one or more sub-components, analytes, ingredients, or constituents of a solution, formulation, or compound relative to one or more other sub-components, analytes, ingredients, or constituents of the solution, formulation, or compound.

The term “solution” can include a mixture of one or more components, analytes, ingredients, or constituents in relative amounts that can be varied. A solution can be a homogenous mixture. A solution can be a heterogenous mixture. A solution can refer to a mixture of components, analytes, ingredients, or constituents in any state of matter.

The term “formulation” can include a material, solution, mixture, or compound that is prepared according to a particular formula or recipe. A formulation can refer to a medicinal or therapeutic preparation administered in a specific form, such as drop, tablet, linctus, ointment, injection, or other formulations.

The term “protein” can include a molecule, macromolecule, or biomolecule made up of, or comprising, amino acids.

The term “therapeutic protein” can refer to proteins that are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

The term “protein solution” can include a mixture of one or more components, analytes, ingredients, or constituents in relative amounts that can be varied; wherein one or more components, analytes, ingredients, or constituents are a molecule, macromolecule, or biomolecule made up of, or comprising, amino acids. A protein solution can be a homogenous mixture. A protein solution can be a heterogenous mixture. A solution can refer to a mixture of components, analytes, ingredients, or constituents in any state of matter.

The term “viscous protein solution” can include a mixture of one or more components, analytes, ingredients, or constituents in relative amounts that can be varied; wherein one or more components, analytes, ingredients, or constituents are a molecule, macromolecule, or biomolecule made up of, or comprising, amino acids; and wherein the mixture has a viscosity of, or of about, 400 to 4000 mPa.s or cP. In some embodiments, the mixture has a viscosity of, or of about, 250 to 4000 mPa.s or cP. Alternatively, a “viscous protein solution” can refer to a comparison of solutions with differing viscosities, in which case, the viscous protein solution can be said to be the solution having the higher viscosity.

The term “viscosity” can refer to the measure of a substance's resistance to motion under an applied force. Viscosity can be determined by dividing the force per unit area required to move one layer of fluid in relation to another (sheer stress) by the measure of the change in speed at which intermediate layers move with respect to one another (sheer rate). Viscosity can be expressed in centipoise (cP). 1 cP is the equivalent of 1 millipascal second (mPa.s).

The term “pascal” can refer to the standard unit of pressure or stress in the International System of Units (SI), equal to one newton per square meter.

The term “evaporative concentration” can refer concentrating one or more sub-components, analytes, ingredients, or constituents of a solution, formulation, or compound by the process of evaporation of one or more other sub-components, analytes, ingredients, or constituents of the solution, formulation, or compound.

The term “evaporation” can refer to a process by which an molecule transitions from a liquid to gaseous state of matter.

The term “boiling point” can refer to the temperature at which the pressure exerted by a liquid's surroundings equals the vapor pressure of the liquid. Application of heat under these conditions can result in transformation of the liquid into a gaseous state without raising the temperature of the liquid.

The term “vapor pressure” can refer to the pressure exerted by a vapor on its condensed phases at a given temperature in a closed system.

The term “relative volatility” can refer to the likelihood of one component to boil vs another in a closed system of two components at a fixed temperature and pressure.

The term “vacuum” can refer to a volume of empty matter. A vacuum can refer to pressures below atmospheric pressure. Reducing the pressure of a closed system containing a solution, formulation, mixture, or compound, can reduce the boiling point of one or more sub-components, analytes, ingredients, or constituents of the solution, formulation, or compound relative to one or more other sub-components, analytes, ingredients, or constituents of the solution, formulation, or compound.

The term “atmospheric pressure,” or barometric pressure, can refer to the force exerted on a surface by the weight of the air above the surface.

The term “patient” can include human and other mammalian subjects that receive either prophylactic or therapeutic treatment.

The terms “therapeutic formulation” or “therapeutic solution” can refer to formulations or solutions that are administered to a patient in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

The term “pharmaceutically acceptable carrier” can refer to an excipient that can be included in compositions and that causes no significant adverse toxicological effect on the patient and is approved or approvable by the FDA for therapeutic use, particularly in humans. Non-limiting examples of pharmaceutically acceptable excipients can include water, NaCl, normal saline solutions, lactated Ringer's, normal sucrose, normal glucose and the like.

The term “antibody” can include intact antibodies and binding fragments thereof. A binding fragment refers to a molecule other than an intact antibody that includes a portion of an intact antibody that binds the antigen to which the intact antibody binds. Examples of binding fragments include Fv, Fab′, Fab′-SH, F(ab′)2; diabodies; linear antibodies; single-chain antibody molecules (e.g. scFv); and multi-specific antibodies formed from antibody fragments. scFv antibodies are described in Houston JS. 1991. Methods in Enzymol. 203:46-96. In addition, antibody fragments include single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N. Y., 1989), Ch. 7) (incorporated by reference in its entirety for all purposes).

The mature variable regions of each light/heavy chain pair form the antibody binding site. Thus, an intact antibody has two binding sites, i e., is divalent. In natural antibodies, the binding sites are the same However, bispecific antibodies can be made in which the two binding sites are different (see, e.g., Songsivilai S, Lachmann PC. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148:1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains include the domains FR1, CDR1, FR2, CDR2, FR3, CDR3 and FR4. For convenience, the variable heavy CDRs can be referred to as CDRH1, CDRH2 and CDRH3; the variable light chain CDRs can be referred to as CDRL1, CDR12 and CDRL3. The assignment of amino acids to each domain is in accordance with the definitions of Kabat E A, et al. 1987 and 1991. Sequences of Proteins of Immunological Interest (National Institutes of Health, Bethesda, M D) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, can be missing or derivatized in a proportion or all of the molecules.

The term “polymer” can refer to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

“Phosphorylcholine,” also denoted as “PC,” refers to the following:

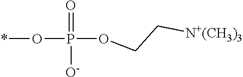

where denotes the point of attachment. The phosphorylcholine is a zwitterionic group and includes salts (such as inner salts), and protonated and deprotonated forms thereof.

A “Phosphorylcholine containing polymer” can refer to a polymer that contains phosphorylcholine. “Zwitterion containing polymer” refers to a polymer that contains a zwitterion.

Poly(acryloyloxyethyl phosphorylcholine) containing polymer can refer to a polymer containing 2-(acryloyloxy)ethyl-2-(trimethylammonium)ethyl phosphate (HEA-PC shown below in Example 6) as monomer.

Poly(methacryloyloxyethyl phosphorylcholine) containing polymer can refer to a polymer containing 2-(methacryloyloxy) ethyl-2-(trimethylammonium ethyl phosphate (HEMA-PC or MPC) as monomer (see below):

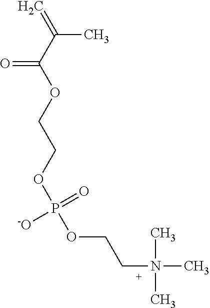

As used herein, “MPC” and “HEMA-PC” are interchangeable.

The term “linear” in reference to the geometry, architecture or overall structure of a polymer, can refer to polymer having a single polymer arm.

The term “branched,” in reference to the geometry, architecture or overall structure of a polymer, can include a polymer having 2 or more polymer “arms” extending from a core structure contained within an initiator. The initiator can be employed in an atom transfer radical polymerization (ATRP) reaction. A branched polymer can possess 2 polymer chains (arms), 3 polymer arms, 4 polymer arms, 5 polymer arms, 6 polymer arms, 7 polymer arms, 8 polymer arms, 9 polymer arms or more. Each polymer arm extends from a polymer initiation site. Each polymer initiation site is capable of being a site for the growth of a polymer chain by the addition of monomers. For example and not by way of limitation, using ATRP, the site of polymer initiation on an initiator is typically an organic halide undergoing a reversible redox process catalyzed by a transition metal compound such as cuprous halide. In some embodiments, the halide is a bromine.

The term “molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation or viscometry to determine weight average molecular weight. In some embodiments, the molecular weight is measured by size exclusion chromatography—multi angle light scattering (hereinafter “SEC-MALS”). In some embodiments, the polymeric reagents are typically polydisperse (i.e., number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

Multi-angle light scattering (MALS) is a technique of analyzing macromolecules where the laser light impinges on the molecule, the oscillating electric field of the light induces an oscillating dipole within it. This oscillating dipole will re-radiate light and can be measured using a MALS detector such as Wyatt miniDawn TREOS. The intensity of the radiated light depends on the magnitude of the dipole induced in the macromolecule which in turn is proportional to the polarizability of the macromolecule, the larger the induced dipole, and hence, the greater the intensity of the scattered light. Therefore, in order to analyze the scattering from a solution of such macromolecules, one should know their polarizability relative to the surrounding medium (e.g., the solvent). This can be determined from a measurement of the change, Δn, of the solution's refractive index n with the molecular concentration change, Δc, by measuring the dn/dc (=Δn/Δc) value using a Wyatt Optilab T-rEX differential refractometer. Two molar weight parameters that MALS determination employ are number average molecular weight (Mn) and weight average molecular weight (Mw) where the polydispersity index (PDI) equals Mw divided by Mn SEC also allows another average molecular weight determination of the peak molecular weight Mp which is defined as the molecular weight of the highest peak at the SEC.

The PDI is used as a measure of the broadness of a molecular weight distribution of a polymer and bioconjugate which is derived from conjugation of a discrete protein (e.g. OG1950) to a polydisperse biopolymer (e.g., OG1802). For a protein sample, its polydispersity is close to 1.0 due to the fact that it is a product of translation where every protein molecule in a solution is expected to have almost the same length and molar mass. In contrast, due to the polydisperse nature of the biopolymer where the various length of polymer chains are synthesized during the polymerization process, it is very important to determine the PDI of the sample as one of its quality attribute for narrow distribution of molecular weight.

Size exclusion chromatography (SEC) is a chromatography technique in which molecules in solution are separated by their size. Typically an aqueous solution is applied to transport the sample through the column which is packed with resins of various pore sizes. The resin is expected to be inert to the analyte when passing through the column and the analytes separate from each other based on their unique size and the pore size characteristics of the selected column.

Coupling the SEC with MALS or SEC/MALS provides accurate distribution of molar mass and size (root mean square radius) as opposed to relying on a set of SEC calibration standards. This type of arrangement has many advantages over traditional column calibration methods. Since the light scattering and concentration are measured for each eluting fraction, the molar mass and size can be determined independently of the elution position. This is particularly relevant for species with non-globular shaped macromolecules such as the biopolymers (OG1802) or bioconjugates (e.g., KSI-301); such species typically do not elute in a manner that might be described by a set of column calibration standards.

In some embodiments, a SEC/MALS analysis includes a Waters HPLC system with Alliance 2695 solvent delivery module and Waters 2996 Photodiole Array Detector equipped with a Shodex SEC-HPLC column (7.8x300mm). This is connected online with a Wyatt miniDawn TREOS and Wyatt Optilab T-rEX differential refractometer. The Empower software from Waters can be used to control the Waters HPLC system and the ASTRA V 6.1.7.16 software from Wyatt can be used to acquire the MALS data from the Wyatt miniDawn TREOS, dn/dc data from the T-rEX detector and the mass recovery data using the A280 absorbance signal from the Waters 2996 Photodiole Array detector. SEC can be carried out at 1ml/min in 1xPBS pH 7.4, upon sample injection, the MALS and RI signals can be analyzed by the ASTRA software for determination of absolute molar mass (Mp, Mw, Mn) and polydisperse index (PDI). In addition, the calculation also involves the input dn/dc values for polymer and protein as 0.142 and 0.183, respectively. For KSI-301 dn/dc value, the dn/dc is calculated based on the weighted MW of the polymer and the protein to be about 0.148 using the formula below:

$$Conjugate dn / dc = 0.142 \times [ MW polymer / ( MW polymer + MW protein ) ] + 0.183 \times [ MW protein / ( MW p olymer + MW protein ) ]$$

where MWpolymer for OG1802 is 800 kDa and the MWprotein for OG1950 is 146 kDa.

Compositions or methods “comprising” one or more recited elements can include other elements not specifically recited. For example, a composition that comprises antibody can contain the antibody alone or in combination with other ingredients.

The phrase “a” or “an” entity can refer to one or more of that entity; for example, a compound can refer to one or more compounds or at least one compound. As such, the terms “a” (or “an”), “one or more”, and “at least one” can be used interchangeably herein.

“About” means variation one might see in measurements taken among different instruments, samples, and sample preparations.

### Methods of Concentrating a Solution or Therapeutic Formulation

Some aspects of the present disclosure are directed towards a method of concentrating a viscous protein solution, the method comprising: providing a first solution comprising protein; and reducing a volume of the first solution via evaporative concentration, wherein a vacuum is applied during evaporative concentration of at least below 100 mbar, to thereby produce a second solution, wherein the second solution has a viscosity between 400 to 4000 mPa.s or cP.

Some aspects of the present disclosure are directed towards a method of concentrating a viscous protein solution, the method comprising: providing a first solution comprising protein; and reducing a volume of the first solution via evaporative concentration, wherein a vacuum is applied during evaporative concentration of at least below 100 mbar, to thereby produce a second solution, wherein the second solution has a viscosity between 250 to 4000 mPa.s or cP.

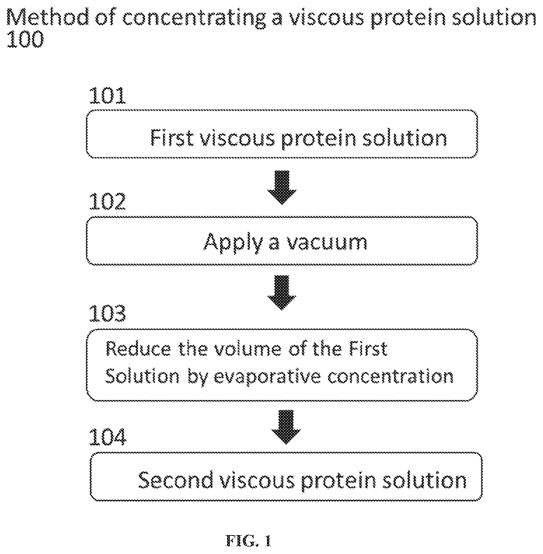

FIG. 1 is a flowchart showing some embodiments of a method of concentrating a viscous protein solution 100. Some embodiments shown in FIG. 1 include providing a first solution 101. In some embodiments, the protein is a therapeutic protein. In some embodiments, the first solution is a protein or therapeutic protein solution, formulation, mixture, or compound. In some embodiments, the protein is a fusion protein. In some embodiments, the protein includes an antibody. In some embodiments, the antibody is a conjugated antibody. In some embodiments, the antibody is an antagonistic antibody. In some embodiments, the conjugated antibody that is provided in the first solution is conjugated to a polymer. In some embodiments, the fusion protein is conjugated to a polymer. In some embodiments, the viscosity of the first solution is known. In some embodiments, the first solution is provided in a vessel. In some embodiments the vessel is pressurized. In some embodiments the vessel is a reactor, chemical reactor, batch flow reactor, continuous flow reactor, or any other vessel suitable for use with the methods disclosed herein. In some embodiments, the pressure is vacuum pressure. In some embodiments, a vacuum pressure is applied to the first solution 102. In some embodiments, the first solution may be agitated, stirred, or mixed prior to, during, and/or after application of the vacuum. In some embodiments, the vacuum is increased to the target vacuum at a constant rate. In some embodiments, the vacuum is increased at different rates throughout the disclosed methods. In some embodiments, the vacuum is established over a desired time. In some embodiments, the vacuum is established over a time sufficient to reduce foaming of the first solution. In some embodiments, application of a vacuum pressure to the first solution lowers the boiling point of one or more sub-components, analytes, ingredients, or constituents of the first solution. In some embodiments, the temperature of the vessel, and or first solution is adjustable. In some embodiments, the temperature of the first solution is adjusted to a temperature that is above the boiling point of one or more sub-components, analytes, ingredients, or constituents of the first solution after application of vacuum pressure to the first solution. In some embodiments, this results in evaporation of one or more sub-components, analytes, ingredients, or constituents of the first solution. In some embodiments, the temperature of the vessel is at the reduced boiling point of one or more sub-components, analytes, ingredients, or constituents of the first solution, such that after application of vacuum pressure to the first solution, one or more sub-components, analytes, ingredients, or constituents of the first solution will evaporate. In some embodiments, the one or more evaporated sub-components, analytes, ingredients, or constituents of the first solution are vented, released, or removed, and/or prevented from reentering the first solution. In some embodiments, this evaporative concentration 103 of the first solution is allowed to proceed for a desired time. In some embodiments, evaporative concentration of the first solution is allowed to proceed until a desired parameter of the first solution has reached. In some embodiments, the desired parameter is viscosity, volume, or the concentration of one or more sub-components, analytes, ingredients, or constituents of the first solution. In some embodiments, this evaporative concentration of the first solution is allowed to continue until the first solution has reached a desired viscosity. In some embodiments, the evaporative concentration of the first solution is allowed to continue until the first solution has reached a desired volume. In some embodiments, the evaporative concentration of the first solution is allowed to continue until the concentration of one or more sub-components, analytes, ingredients, or constituents of the first solution has reached a desired concentration. Removal of the one or more evaporated sub-components, analytes, ingredients, or constituents of the first solution thereby results in the production of a second solution 104. In some embodiments, the second solution has a desired viscosity, volume, or concentration of one or more sub- components, analytes, ingredients, or constituents of the second solution. In some embodiments, the disclosed methods further include processing of the second solution or formulation. In some embodiments, processing of the second solution includes venting the second volume. In some embodiments, processing of the second solution includes filtering the concentrated viscous protein solution. In some embodiments, processing of the second solution includes collecting quality control samples during the evaporative concentration process. In some embodiments, processing of the second solution includes storage of the concentrated viscous protein solution.

Some aspects of the present disclosure are directed towards a method of reducing a volume of a therapeutic formulation, the method comprising: providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution; wherein the second solution has a viscosity between 400 to 4000 mPa.s or cP.

Some aspects of the present disclosure are directed towards a method of reducing a volume of a therapeutic formulation, the method comprising. providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution, wherein the second solution has a viscosity between 250 to 4000 mPa.s or cP.

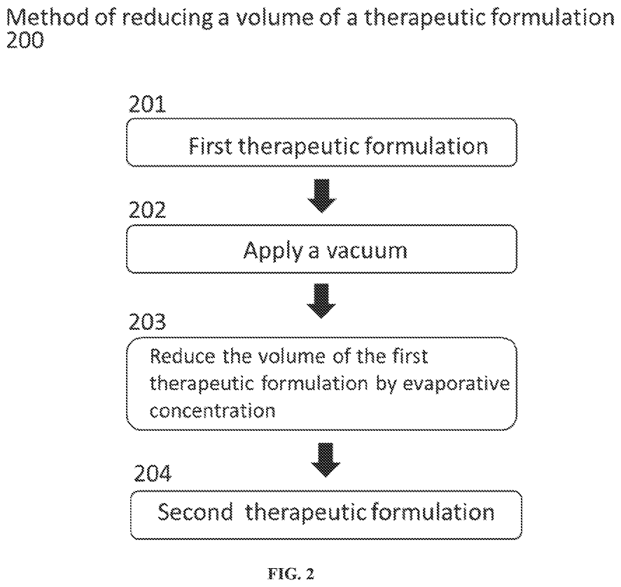

FIG. 2 is a flowchart showing some embodiments of a method of a method of reducing a volume of a therapeutic formulation 200. In some embodiments, the method comprises providing a first solution comprising a conjugated polymer 201. In some embodiments, the polymer is conjugated to a protein. In some embodiments, the protein is an antibody. In some embodiments the protein is a fusion protein. In some embodiments, the protein is a therapeutic protein, therapeutic fusion protein, therapeutic antibody, or therapeutic conjugated antibody. In some embodiments, the first solution is at a desired first volume. In some embodiments, a vacuum pressure of at least below 100 mbar is applied to the first solution 202. In some embodiments, application of a vacuum pressure to the first solution lowers the boiling point of one or more sub-components, analytes, ingredients, or constituents of the first solution. In some embodiments, the first solution is at, or is adjusted to, a temperature above the reduced boiling point of one or more sub-components, analytes, ingredients, or constituents of the first solution, resulting in evaporation of one or more sub-components, analytes, ingredients, or constituents of the first solution. In some embodiments, the one or more evaporated sub-components, analytes, ingredients, or constituents of the first solution are vented, released, or removed, and/or prevented from reentering the first solution, thereby reducing the volume of the first solution to a second volume 203. In some embodiments, this evaporative concentration of the first solution is allowed to continue until the first solution has reached a desired volume, or viscosity. In some embodiments, evaporative concentration of the first solution is allowed to continue until one or more sub-components, analytes, ingredients, or constituents of the first solution reach a desired concentration. Removal of the one or more evaporated sub-components, analytes, ingredients, or constituents of the first solution thereby results in the production of a second solution 204. In some embodiments, the second solution has a desired viscosity, volume, or concentration of one or more sub-components, analytes, ingredients, or constituents of the second solution.

Some aspects of the present disclosure are directed towards a method of reducing a volume of a therapeutic formulation, the method comprising: providing a first solution, the first solution comprising at least 0.001% polysorbate and a conjugate, wherein the conjugate comprises a polymer conjugated to an antibody or a small-molecule drug or both, wherein the first solution is at a first volume, and applying a vacuum of at least below 100 mbar to the first solution to reduce the first volume to a second volume by evaporative concentration to thereby produce a second solution, wherein the second solution has a viscosity between 400 to 4000 mPa.s or cP, wherein evaporative concentration is carried out at less than 40° C., and wherein the vacuum of at least below 100 mbar is established over at least 15 minutes to reduce foaming.

Some aspects of the present disclosure are directed towards a method of reducing a volume of a therapeutic formulation, the method comprising: providing a first solution, the first solution comprising at least 0.001% polysorbate and a conjugate, wherein the conjugate comprises a polymer conjugated to an antibody or a small-molecule drug or both, wherein the first solution is at a first volume; and applying a vacuum of at least below 100 mbar to the first solution to reduce the first volume to a second volume by evaporative concentration to thereby produce a second solution, wherein the second solution has a viscosity between 250 to 4000 mPa.s or cP, wherein evaporative concentration is carried out at less than 40° C., and wherein the vacuum of at least below 100 mbar is established over at least 15 minutes to reduce foaming.

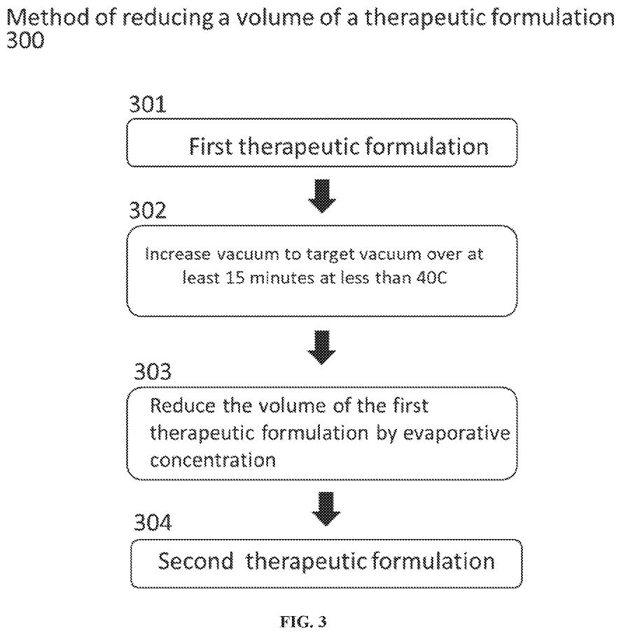

FIG. 3 is a flowchart showing some embodiments of a method of reducing a volume of a therapeutic formulation 300. In some embodiments, the method includes providing a first solution comprising a conjugated polymer and a surfactant 301. In some embodiments the polymer is conjugated to a protein, fusion protein, therapeutic protein, antibody, therapeutic antibody, or small molecule drug. In some embodiments, the surfactant is at a desired concentration. In some embodiments the surfactant is a polysorbate. In some embodiments, the first solution is provided in a vessel. In some embodiments the vessel is pressurized. In some embodiments the vessel is a reactor, chemical reactor, batch flow reactor, continuous flow reactor, or any other vessel suitable for use with the methods disclosed herein. In some embodiments, the first solution is at a desired first volume. In some embodiments, a vacuum pressure of at least below 100 mbar is applied to the first solution. In some embodiments, the vacuum is increased at a constant rate. In some embodiments, the vacuum is increased at different rates throughout the disclosed methods. In some embodiments, the vacuum is established over a desired time. In some embodiments, the vacuum is established over a time sufficient to reduce foaming of the first solution. In some embodiments, application of a vacuum pressure to the first solution results in evaporative concentration of the first solution. In some embodiments evaporative concentration of the first solution is carried out at a desired temperature. In some embodiments, the temperature of the vessel, and or first solution is adjustable. In some embodiments, the temperature of the first solution is adjusted to a temperature that is above the boiling point of one or more sub-components, analytes, ingredients, or constituents of the first solution after application of vacuum pressure to the first solution. In some embodiments, the temperature of the vessel is at the reduced boiling point of one or more sub-components, analytes, ingredients, or constituents of the first solution, such that after application of vacuum pressure to the first solution, one or more sub-components, analytes, ingredients, or constituents of the first solution will evaporate. Venting of the one or more evaporated sub-components, analytes, ingredients, or constituents of the first solution results in the production of a second solution 304 by evaporative concentration of the first solution 303. In some embodiments, evaporative concentration of the first solution is allowed to continue until the first solution has reached a desired viscosity. In some embodiments, evaporative concentration of the first solution is allowed to continue until one or more sub-components, analytes, ingredients, or constituents of the first solution reach a desired concentration. In some embodiments, the second solution has a desired viscosity, volume, or concentration of one or more sub-components, analytes, ingredients, or constituents of the second solution.

In some embodiments, the protein solution comprises a surfactant. In some embodiments, the protein solution comprises polysorbate. In some embodiments, the protein solution comprises Polysorbate-20, 40, 60, 65, and/or 80. In some embodiments, the first protein solution comprises, comprises about, comprises at least, comprises at least about, comprises not more than, or comprises not more than about, 0.0005%, 0.001%, 0.002%, 0.003%, 0.004%, 0.005%, 0.006%, 0.007%, 0.008%, 0.009%, 0.01%, 0.125%, 0.025%, 0.05%, 0.1%, 0.11% 0.12%, 0.13%, 0.14%, 0.15%, 0.16%, 0.17%, 0.18%, 0.19%, 0.20%, 0.21%, 0.22%, 0.23%, 0.24%, 0.25%, 0.3%, 0.4%, or 0.5% surfactant, or is range defined by any two of the preceding values. For example, in some embodiments, the concentration of surfactant is 0.0005% to 0.5%, 0.0005% to 0.25%, 0.0005%, to 0.1%, 0.0005% to 0.01%, 0.001% to 0.5%, 0.001% to 0.25%, 0.001% to 0.1%, or 0.001% to 0.01% surfactant. In some embodiments, the surfactant includes an anionic, cationic, amphoteric, zwitterionic, and/or non-ionic surfactant. In some embodiments, the surfactant includes ethylene, propylene oxide, fatty acid esters, sorbitol, sorbitol derivatives, sorbitan esters, ethoxylates, and/or copolymers. In some embodiments the surfactant includes polysorbate. In some embodiments the surfactant includes Polysorbate-20, Polysorbate-40, Polysorbate-60, Polysorbate-65, and/or Polysorbate-80. In some embodiments the surfactant includes a single polysorbate. In some embodiments, the surfactant includes multiple polysorbates. In some embodiments, the surfactant includes a single surfactant. In some embodiments, the surfactant includes a combination of surfactants.

In some embodiments, a volume of the first solution is reduced via evaporative concentration. In some embodiments, the volume of the first solution is reduced by a desired volume. In some embodiments, the volume of the first solution is reduced by about, 20, 25, 30, 35, 40, 45, 50, 55, 60, 65, 70, 75, or 80%, or by a range defined by any two of the preceding values. For example, in some embodiments, the volume of the first solution is reduced by about, 20 to 80%, 20 to 50%, 30 to 80%, or 30 to 50%. In some embodiments, the volume of the first solution is reduced to a specific concentration of one or more analytes in the protein solution. In some embodiments, the analyte is protein concentration. In some embodiments, the analyte is surfactant concentration. In some embodiments, the vacuum is applied to the first solution until the concentration of an analyte in the first solution has been concentrated by about 0.1, 0.5, 1, 1.5, 2, 3, 4, 4.5, or 5-fold, or by a range defined by any two of the preceding values. For example, in some embodiments, the concentration of an analyte in the first protein solution is concentrated by about 0 1 to 5-fold, 0.1 to 3-fold, 1-5 fold, or 2-5 fold. In some embodiments, evaporative concentration of the first protein solution results in an increased, decreased, or similar viscosity of the first protein solution.

In some embodiments, a vacuum is applied during evaporative concentration to reduce the volume of the first protein solution. In some embodiments, the vacuum applied is about 13, 17, 26, 50, 51, 80, 81, 100, 101, 111, 112, 150, 151, 200, 202, or 203 mbar, or is range defined by any two of the preceding values. For example, in some embodiments, the vacuum applied is about 13 to 203 mbar, 13 to 101 mbar, 13 to 100 mbar, 17 to 203 mbar, 17 to 101 mbar, or 17 to 100 mbar. In some embodiments, the vacuum applied is below at least about 100 mbar. In some embodiments, the vacuum applied is about, 80, 85, 86, 87, 88, 89, 90, 91, 92, 93, 94, 95, 96, 97, 98, or 99% vacuum, or is range defined by any two of the preceding values. For example, in some embodiments, the vacuum applied is about 80.0 to 100%, 80.0 to 98.7%, 80.0 to 97.4%, 90% to 100%, 90% to 98.7%, 90% to 97.4%, or 97.4 to 98.7% vacuum. In some embodiments, the vacuum is established over a desired length of time. In some embodiments, the vacuum is increased from atmospheric pressure to the desired vacuum over a period of, or of about, 0, 10, 15, 30, 45, 60, 90, 120, 150, 180, 240, 300, or 360 minutes, or is range defined by any two of the preceding values. For example, in some embodiments, the vacuum is increased from atmospheric pressure to the desired vacuum over a period of, or of about, 0 to 360, 0 to 180, 0 to 90, 30 to 360, 30 to 180, or 30 to 90 minutes. In some embodiments, the vacuum is increased in stages, steps, or incrementally. In some embodiments, the vacuum is increased from atmospheric pressure to lower than about, 13, 14, 15, 16, 17, 18, 19, 20, 21, 22, 23, 24, 25, 26, or 27 mbar at a rate of about 75, 80, 85, 86, 87, 88, 89, 90, 91, 92, 93, 94, 95, 100, 110, 125, or 150 mbar/min, or at rate in a range defined by any two of the preceding values. For example, in some embodiments, the vacuum is increased from atmospheric pressure to lower than 13 to 27 mbar at a rate of 75 to 150, 75 to 100, 85 to 150, or 85 to 100 mbar/min. In some embodiments, the vacuum is increased from between, or between about, 75 and 110 mbar to the desired vacuum at a rate of between, or between about 0.1 and 2.0 mbar/min. In some embodiments, the vacuum is increased from between about, 75 and 110 mbar to the desired vacuum at a rate of, or of about, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mbar/min or at rate in a range defined by any two of the preceding values. For example, in some embodiments, the vacuum is increased from about 75 to 110 mbar to the desired vacuum at a rate of about, 0.01 to 2.0, 1.0 to 1.5, 0.5 to 2.0, 0.5 to 1.5 mbar/min. In some embodiments, the vacuum is increased from about 100 mbar to the desired vacuum at a rate of about, 1.0 mbar/min. In some embodiments, the vacuum is applied for a desired length of time. For example, in some embodiments, the vacuum is applied to the first solution for about 0, 6, 12, 18, 24, 20, 36, 42, 48, 54, 60, 66, 72, 78, 84, or 90 hours, or for a range defined by any two of the preceding values. For example, in some embodiments, the vacuum is applied to the first solution for between about 0 and 96 hours, 0 and 72 hours, 0 and 48 hours, 24 and 96 hours, 24 and 72 hours, 24 and 48 hours, or 48 and 72 hours. As used herein, when a “vacuum is increased” it means that there is a decrease in pressure.

In some embodiments, the vacuum can be adjusted if the temperature is adjusted accordingly. Table 1 list various vacuum pressures that may be used in the disclosed processes at different temperatures. For example, in some embodiments, evaporative concentration is carried out at a vacuum pressure of 10 mbar and a temperature of 7° C. In some embodiments, evaporative concentration is carried out at a vacuum pressure of 17 mbar and a temperature of 15° C. In some embodiments, evaporative concentration is carried out at a vacuum pressure of 24 mbar and a temperature of 21° C. In some embodiments, evaporative concentration is carried out at a vacuum pressure of 30 mbar and a temperature of 24° C. In some embodiments, evaporative concentration is carried out at a vacuum pressure of 42 mbar and a temperature of 30° C. In some embodiments, evaporative concentration is carried out at a vacuum pressure of 73 mbar is used and a temperature of 40° C. In some embodiments, a vacuum pressure of 123 mbar is used at a temperature of 50° C.

TABLE 1

| Vacuum | Temperature |
|---|---|
| 10 mbar | 7° C. |
| 17 mbar | 15° C. |
| 24 mbar | 21° C. |
| 30 mbar | 24° C. |
| 42 mbar | 30° C. |
| 73 mbar | 40° C. |
| 123 mbar | 50° C. |

In some embodiments, the result of evaporative concentration of the first solution is the production of a second solution. In some embodiments, the second protein solution has a viscosity of about 400, 500, 600, 700, 750, 800, 900, 1000, 1100, 1200, 1250, 1300, 1400, 1500, 1600, 1700, 1800, 1900, 2000, 2500, 3000, 3500, or 4000 mPa.s or cP, or a viscosity in a range defined by any two of the preceding values. For example, in some embodiments, the viscosity of the second solution is about 400 to 4000, 400 to 3500, 400 to 3000, 400 to 2500, 500 to 2500, 500 to 5000, 600 to 2500, 600 to 4000, 700 to 2500, or 700 to 4000 mPa.s or cP. In some embodiments, the second protein solution has a viscosity of about 250, 300, 400, 500, 600, 700, 750, 800, 900, 1000, 1100, 1200, 1250, 1300, 1400, 1500, 1600, 1700, 1800, 1900, 2000, 2500, 3000, 3500, or 4000 mPa.s or cP, or a viscosity in a range defined by any two of the preceding values. In some embodiments, the viscosity of the second solution is about 250 to 4000, 250 to 3500, 250 to 3000, 250 to 2500, 300 to 2500, 300 to 1500, or 300 to 400 mPa.s or cP. In some embodiments, the second solution is a therapeutic protein solution or formulation. In some embodiments, the concentration of the protein in the second solution is about 0, 10, 20, 30, 40, 41, 42, 43, 44, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 49, 50, 51, 52, 53, 54, 55, 56, 57, 58, 59, 60, 65, 70, 75, 80, 85, 90, 95, 100, 125, 150, or 200 mg/mL, or is a range defined by any two of the preceding values. For example, in some embodiments, the concentration of the protein in the second solution is about, 0 to 200, 0 to 150, 0 to 100, 0 to 75, 0 to 50, 0 to 25, 10 to 200, 10 to 150, 10 to 100, 10 to 50, 10 to 25, 30 to, 30 to 150, 30 to 100, or 30 to 50 mg/mL.

In some embodiments, the temperature of the first and/or second solution is maintained at a desired temperature for some or all of the disclosed methods. In some embodiments, the temperature of the first and/or second solution is maintained at the same temperature for some or all of the disclosed method. In some embodiments, the temperature of the first and/or second solution is maintained at different temperatures for some or all of the disclosed method. In some embodiments, the temperature of the first and or second solution is maintained at a temperature between about, the boiling point and freezing point of the first and/or second solutions. In some embodiments, the temperature of the first and/or second solution is maintained at about 0, 1, 2, 3, 4, 5, 6, 7, 8, 9, 10, 15, 20, 25, 30, 35, 40, 45, 50 60, 70, 80, 90, or 100 C, or at a range defined by any two of the preceding values. For example, in some embodiments, the temperature of the first and/or second solution is about, 0 to 100 C, 0 to 80 C, 0 to 50 C, 0 to 20 C, 20 to 100 C, 20 to 80 C, or 20 to 50 C. In some embodiments, the temperature of the first or second solution is chosen for its suitability in evaporative concentration of one or more sub-components, analytes, ingredients, or constituents of the first and/or second solution.

In some embodiments, the first and/or second solution are agitated for some or all of the duration of the disclosed methods. In some embodiments, the first and/or second solution are agitated for about 0, 15, 30, 45, 60, 90, 120, 150, 180, 240, 300, 360 minutes, or at a range defined by any two of the preceding values. For example, in some embodiments, the first and/or second solution is agitated for about 0 to 360 min, 0 to 240 min, 0 to 180 min, 0 to 120 min, 0 to 90 min, 0 to 60 min, 0 to 30 min, 0 to 15 min, 30 to 360 min, 30 to 240 min, 30 to 120 min, 30 to 90 min, 30 to 60 min, or 15 to 30 min. In some embodiments, the first and/or second solution are agitated for up to about 72 hours. For example, in some embodiments, the first and/or second solution are stirred for up to about 0, 6, 12, 18, 24, 20, 36, 42, 48, 54, 60, 66, 72, 78, 84, or 90 hours, or for a range defined by any two of the preceding values. For example, in some embodiments, the first and/or second solution are agitated for between about 0 and 96 hours, 0 and 72 hours, 0 and 48 hours, 24 and 96 hours, 24 and 72 hours, 24 and 48 hours, or 48 and 72 hours. In some embodiments, the agitation includes stirring, mixing, or other suitable means of agitating a solution, therapeutic solution, formulation, or therapeutic formulation. In some embodiments, the first and/or second solution are agitated, stirred, or mixed, at about 25, 30, 35, 40, 45, 50, 55, 60, 65, 70, 75, 80, 85, 90,95, 100, 125, 150, 175, 200, 250, 300, 350, 400, 450, 500, 600, 700, 800, 900, or 1000 RPM, or at a range defined by any two of the preceding values. For example, in some embodiments, the first and/or second solution are agitated, stirred, or mixed at about 25 to 1000 RPM, 25 to 700 RPM, 25 to 500 RPM, 25 to 250 RPM, 25 to 150 RPM, 25 to 100 RPM, 25 to 50 RPM, 100 to 1000 RPM, 100 to 700 RPM, 100 to 500 RPM, 100 to 250 RPM, or 500 to 1000 RPM. In some embodiments, the first and/or second solution are agitated, stirred, or mixed, at about 15, 25, 30, 35, 40, 45, 50, 55, 60, 65, 70, 75, 80, 85, 90,95, 100, 125, 150, 175, 200, 250, 300, 350, 400, 450, 500, 600, 700, 800, 900, or 1000 RPM, or at a range defined by any two of the preceding values. For example, in some embodiments, the first and/or second solution are agitated, stirred, or mixed at about 15 to 1000 RPM, 15 to 700 RPM, 15 to 500 RPM, 15 to 250 RPM, 15 to 150 RPM, 15 to 100 RPM, 15 to 50 RPM, 100 to 1000 RPM, 100 to 700 RPM, 100 to 500 RPM, 100 to 250 RPM, or 500 to 1000 RPM. In some embodiments, the first and/or second solution are stirred continuously. In some embodiments, the first and/or second solution are stirred intermittently. In some embodiments, the first and/or second solution are stirred to homogeneity.

In some embodiments, the protein solution, therapeutic solution, therapeutic formulation, first solution and/or second solution, as described herein, includes a protein. In some embodiments, the protein includes an antibody. In some embodiments, the antibody is a conjugated antibody. In some embodiments, the antibody is an antagonistic antibody. In some embodiments, the therapeutic solution or formulation is a pharmaceutical composition comprising an isolated antagonistic antibody, and/or a pharmaceutically acceptable carrier. In some embodiments, the therapeutic solution or formulation includes a formulation for the treatment or prophylaxis of an ocular disorder. In some embodiments, the antibody is a conjugated antibody. In some embodiments, the first solution further comprises an unconjugated antibody. In some embodiments, the first solution does not further comprise an unconjugated antibody.

In some embodiments, the conjugated antibody comprises: a CDRH1 that is a CDRH1 in SEQ ID NO: 28; a CDRH1 that is a CDRH1 in SEQ ID NO: 29; a CDRH1 that is a CDRH1 in SEQ ID NO: 30; a CDRH1 that is a CDRH1 in SEQ ID NO: 33; a CDRH1 that is a CDRH1 in SEQ ID NO: 34;a CDRH1 that is a CDRH1 in SEQ ID NO: 35; at least one of the following mutations (EU numbering): L234A, L235A, and G237A; and at least one of the following mutations (EU numbering): Q347C or L443C.

In some embodiments, the conjugated antibody comprises a heavy chain that has a sequence of at least one of SEQ ID NOs: 2-17, 21, 22, 36-42; and a light chain that has a sequence of at least one of SEQ ID NOs: 18-20, 23-25. In some embodiments, the conjugated antibody comprises a CDRH1 that is a CDRH1 in SEQ ID NO: 43; a CDRH2 that is a CDRH2 in SEQ ID NO: 43; and a CDRH3 that is a CDRH3 in SEQ ID NO: 43; and a CDRL1 comprising H31, N33, G34 or E34 or F34 or S34, D35 or E35, T36 or S36 or V36, Y37, L38 or 138, and E39 (EU numbering); a CDRL2 comprising LS1 or H51, 153 or V53, and K55 (EU numbering); and a CDRL3 comprising F94 or L94, Q95, G96, S97, V99 or N99 or Q99 or W99, P100, and P101 or V101 (EU numbering).

In some embodiments, the conjugated antibody comprises a CDRH1 that is a CDRH1 in SEQ ID NO: 43; a CDRH2 that is a CDRH2 in SEQ ID NO: 43; a CDRH3 that is a CDRH3 in SEQ ID NO: 43; a VL comprising: a CDRL1 that is a CDRL1 in SEQ ID NO: 44; a CDRL2 that is a CDRL2 in SEQ ID NO: 44; a CDRL3 that is the CDRL3 in SEQ ID NO: 44; at least one of the following mutations (EU numbering): L234A, L235A, and G237A; and at least one of the following mutations (EU numbering): Q347C or L443C.

In some embodiments, the conjugate has the following structure:

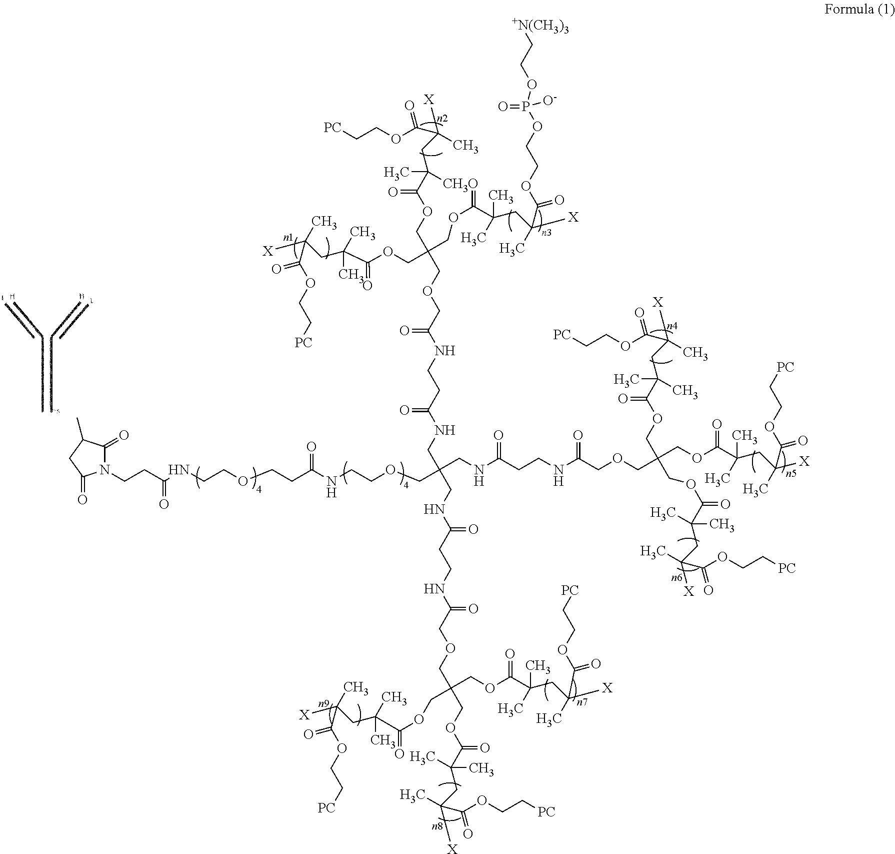

wherein: each heavy chain of the antibody is denoted by the letter H, and each light chain of the antibody is denoted by the letter L; the polymer is bonded to the antibody through the sulfhydryl of C443 (EU numbering), which bond is depicted on one of the heavy chains; PC is

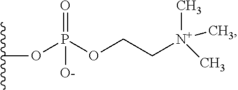 [SMILES: *OP(=O)([O-])OCC[N+](C)(C)C]

where the curvy line indicates the point of attachment to the rest of the polymer, where X=a) OR where R═H, methyl, ethyl, propyl, isopropyl, b) H, or c) any halide, including Br; and n1, n2, n3, n4, n5, n6, n7, n8 and n9 are the same or different such that the sum of n1, n2, n3, n4, n5, n6, n6, n7, n8 and n9 is 2500 plus or minus 15%.

In some embodiments, the protein comprises a fusion protein. In some embodiments, the protein is a fusion protein comprising a vascular endothelial growth factor (hereinafter “VGEF”) antagonist linked to a platelet-derived growth factor (herein after “PDGFR”) extracellular trap segment, wherein the PDGFR extracellular trap segment comprises domains D1-D3 of PDGFR-β.

Polynucleotides complementary to any such sequences are also encompassed by the present invention. Polynucleotides can be single-stranded (coding or antisense) or double-stranded, and can be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide can, but need not, be linked to other molecules and/or support materials.

Polynucleotides can comprise a native sequence (i.e., an endogenous sequence that encodes an antibody or a fragment thereof) or can comprise a variant of such a sequence. Polynucleotide variants can contain one or more substitutions, additions, deletions and/or insertions such that the activity of the encoded polypeptide is not diminished, relative to a native molecule. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence can be compared to a reference sequence of the same number of contiguous positions after the two sequences are optimally aligned.

Optimal alignment of sequences for comparison can be conducted using the MegAlign® program in the Lasergene® suite of bioinformatics software (DNASTAR®, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D.G. and Sharp, P.M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, WI, using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

Preferably, the “percentage of sequence identity” is determined by comparing two optimally aligned sequences over a window of comparison of at least 20 positions, wherein the portion of the polynucleotide or polypeptide sequence in the comparison window can comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants can also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

In some embodiments, the antibody is conjugated to a polymer. In some embodiments, the fusion protein is conjugated to a polymer. In some embodiments, the polymer is covalently bonded to the antibody at a cysteine outside a variable region of the antibody wherein said cysteine has been added via recombinant technology. In some embodiments, the polymer is a biopolymer. In some embodiments, the biopolymer comprises an aflibercept biopolymer. In some embodiments, the polymer comprises a phosphorylcholine polymer. In some embodiments, the phosphorylcholine containing polymer comprises 2-(methacryloyloxyethyl)-2′-(trimethylammonium)ethyl phosphate (MPC) monomers as set forth below:

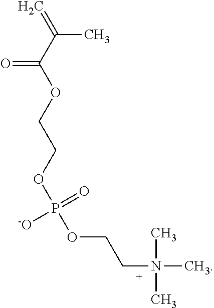

In some embodiments, the polymer has three or more arms or is synthesized with an initiator comprising 3 or more polymer initiation sites. In some embodiments, the polymer has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites. In some embodiments, the polymer has 2, 3, 6, or 9 arms or is synthesized with an initiator comprising 2, 3, 6, or 9 polymer initiation sites. In some embodiments, the polymer has 9 arms or is synthesized with an initiator comprising 9 polymer initiation sites. In some embodiments, the polymer has a molecular weight between, or between about, 100,000 and 3,500,000 Da as measured by SEC-MALS. In some embodiments, the polymer has a molecular weight between, or between about, 100,000 and 2,500,000 Da as measured by SEC-MALS. In some embodiments, the polymer has a molecular weight between, or between about, 100,000 and 1,750,000 Da as measured by SEC-MALS. In some embodiments, the polymer has a molecular weight between, or between about, 300,000 and 3,500,000 Da as measured by SEC-MALS. In some embodiments, the polymer has a molecular weight between, or between about, 300,000 and 2,500,000 Da as measured by SEC-MALS. In some embodiments, the polymer has a molecular weight between, or between about, 300,000 and 1,750,000 Da as measured by SEC-MALS.

In some embodiments, the polymer comprises a zwitterionic monomer. In some embodiments, the zwitterionic monomer comprises one or more of HEMA-phosphorylcholine, PEG, biocompatible fatty acids and derivatives thereof, Hydroxy Alkyl Starch (HAS), Hydroxy Ethyl Starch (HES), Polyethylene Glycol (PEG), Poly (Glyx-Sery) (HAP), Hyaluronic Acid (HA), Heparosan polymers (HEP), Fleximers, Dextran, Poly-sialic acids (PSA), FC domains, Transferrin, 25 Albumin, Elastin Like Peptides (ELP), CTP peptides, and/or FCRn binding peptides.

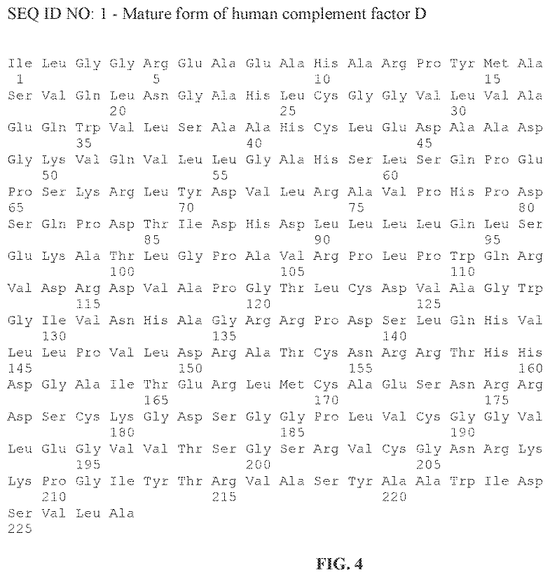

In some embodiments, the first solution comprises any one or more of SEQ ID NOs: 1-50. FIGS. 4-53 show embodiments of polynucleotide sequences of SEQ ID NOs: 1-50.

In some embodiments, the first and or second solution comprises a protein with the polynucleotide sequence of SEQ ID NO: 1. FIG. 4 shows some embodiments of a polynucleotide sequence of a mature form of human complement factor D polynucleotide. (SEQ ID NO: 1).

In some embodiments, the first and or second solution comprises a protein with the polynucleotide sequence of SEQ ID NO: 2. FIG. 5 shows some embodiments of a polynucleotide sequence of a mAb KCD005 heavy chain polynucleotide. (SEQ ID NO: 2).

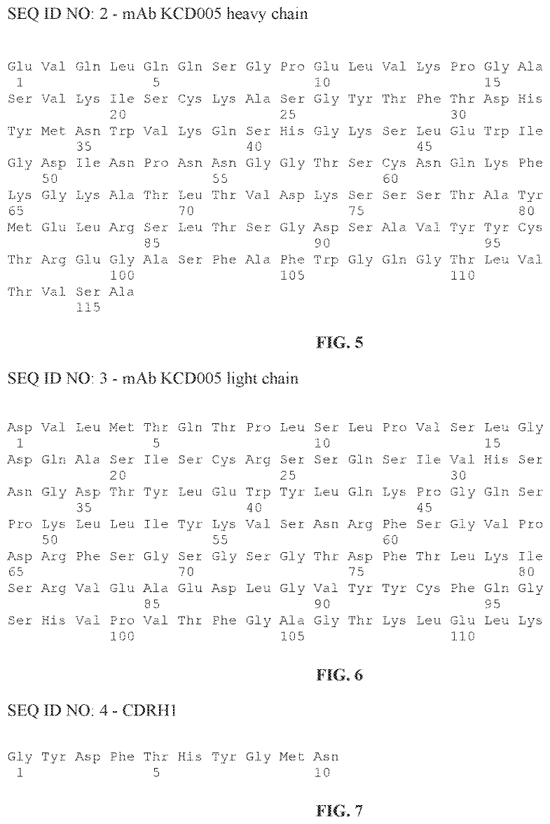

In some embodiments, the first and or second solution comprises a protein with the polynucleotide sequence of SEQ ID NO: 3. FIG. 6 shows some embodiments of a polynucleotide sequence of a mAb KCD005 light chain polynucleotide. (SEQ ID NO: 3).

In some embodiments, the first and or second solution comprises a protein with the polynucleotide sequence of SEQ ID NO: 4. FIG. 7 shows some embodiments of a polynucleotide sequence of a CDRH1 polynucleotide. (SEQ ID NO: 4).

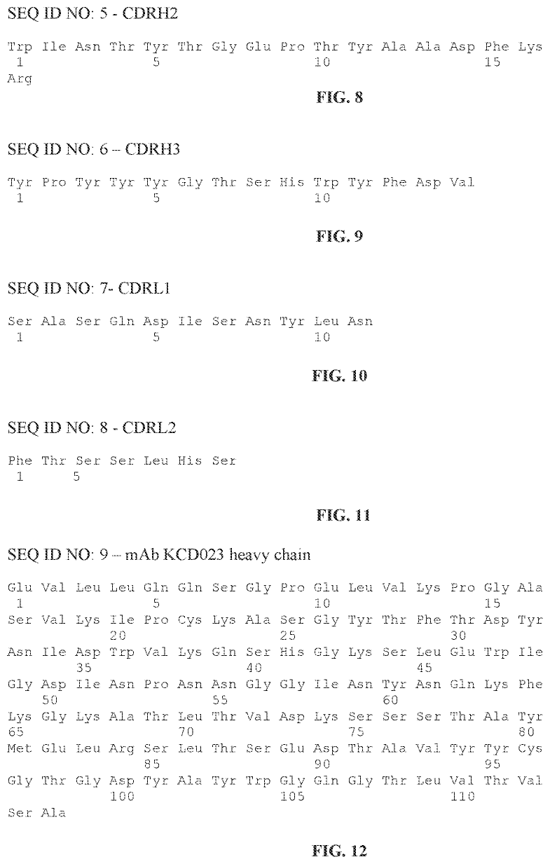

In some embodiments, the first and or second solution comprises a protein with the polynucleotide sequence of SEQ ID NO: 5. FIG. 8 shows some embodiments of a polynucleotide sequence of a CDRH2 polynucleotide. (SEQ ID NO: 5).

In some embodiments, the first and or second solution comprises a protein with the polynucleotide sequence of SEQ ID NO: 6. FIG. 9 shows some embodiments of a polynucleotide sequence of a CDRH3 polynucleotide. (SEQ ID NO: 6).

In some embodiments, the first and or second solution comprises a protein with the polynucleotide sequence of SEQ ID NO: 7. FIG. 10 shows some embodiments of a polynucleotide sequence of a CDRL1 polynucleotide. (SEQ ID NO: 7).

In some embodiments, the first and or second solution comprises a protein with the polynucleotide sequence of SEQ ID NO: 8. FIG. 11 shows some embodiments a polynucleotide sequence of a CDRL2 polynucleotide. (SEQ ID NO: 8).

In some embodiments, the first and or second solution comprises a protein with the polynucleotide sequence of SEQ ID NO: 9. FIG. 12 shows some embodiments of a polynucleotide sequence of a mAb KCD023 heavy chain polynucleotide. (SEQ ID NO: 9).

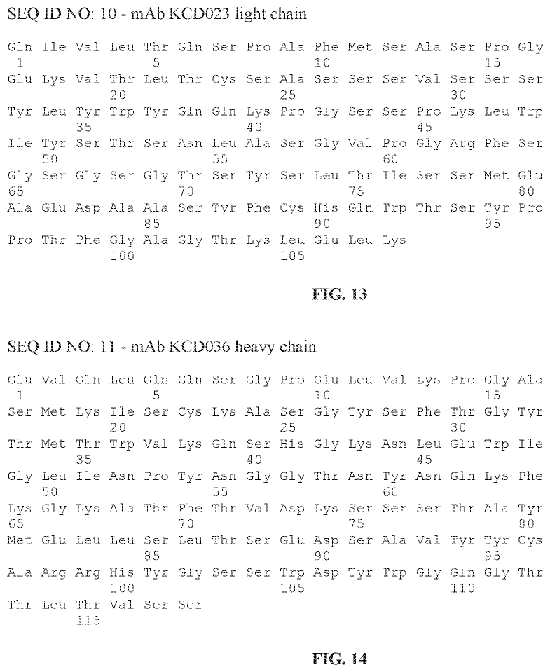

In some embodiments, the first and or second solution comprises a protein with the polynucleotide sequence of SEQ ID NO: 10. FIG. 13 shows some embodiments of a polynucleotide sequence of a mAb KCD023 light chain polynucleotide. (SEQ ID NO: 10).

In some embodiments, the first and or second solution comprises a protein with the polynucleotide sequence of SEQ ID NO: 11. FIG. 14 shows some embodiments of a polynucleotide sequence of a mAb KCD036 heavy chain polynucleotide. (SEQ ID NO: 11).

In some embodiments, the first and or second solution comprises a protein with the polynucleotide sequence of SEQ ID NO: 12. FIG. 15 shows some embodiments of a polynucleotide sequence of a mAb KCD036 light chain polynucleotide. (SEQ ID NO: 12).

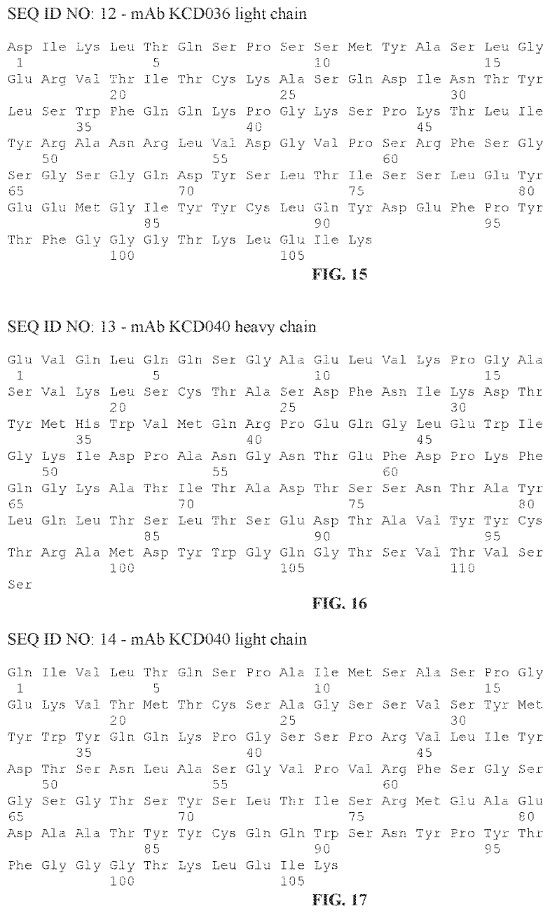

In some embodiments, the first and or second solution comprises a protein with the polynucleotide sequence of SEQ ID NO: 13. FIG. 16 shows some embodiments of a polynucleotide sequence of a mAb KCD040 heavy chain polynucleotide. (SEQ ID NO: 13).

In some embodiments, the first and or second solution comprises a protein with the polynucleotide sequence of SEQ ID NO: 14. FIG. 17 shows some embodiments of a polynucleotide sequence of a mAb KCD040 light chain polynucleotide. (SEQ ID NO: 14).

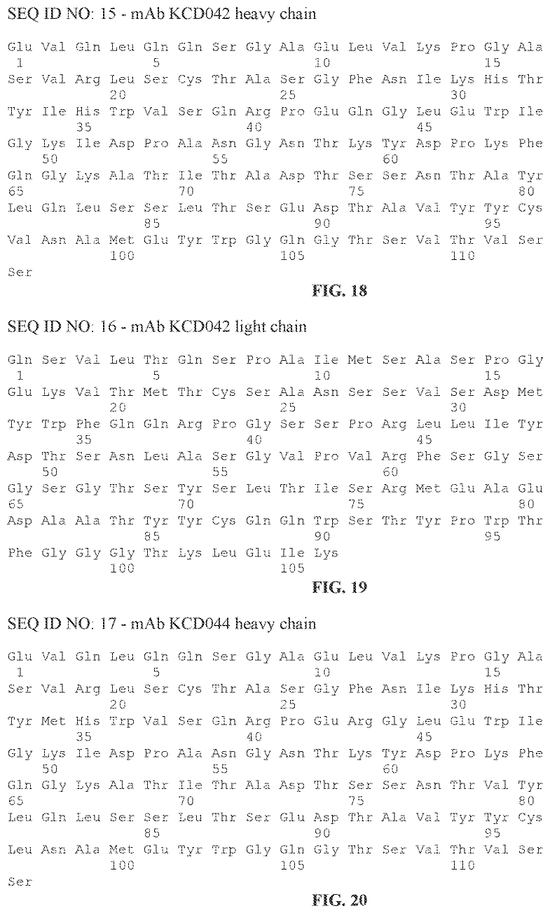

In some embodiments, the first and or second solution comprises a protein with the polynucleotide sequence of SEQ ID NO: 15. FIG. 18 shows some embodiments of a polynucleotide sequence of a mAb KCD042 heavy chain polynucleotide. (SEQ ID NO: 15).

In some embodiments, the first and or second solution comprises a protein with the polynucleotide sequence of SEQ ID NO: 16. FIG. 19 shows some embodiments of a polynucleotide sequence of a mAb KCD042 light chain polynucleotide. (SEQ ID NO: 16).

In some embodiments, the first and or second solution comprises a protein with the polynucleotide sequence of SEQ ID NO: 17. FIG. 20 shows some embodiments of a polynucleotide sequence of a mAb KCD044 heavy chain polynucleotide. (SEQ ID NO: 17).

In some embodiments, the first and or second solution comprises a protein with the polynucleotide sequence of SEQ ID NO: 18. FIG. 21 shows some embodiments of a polynucleotide sequence of a mAb KCD044 light chain polynucleotide. (SEQ ID NO: 18).

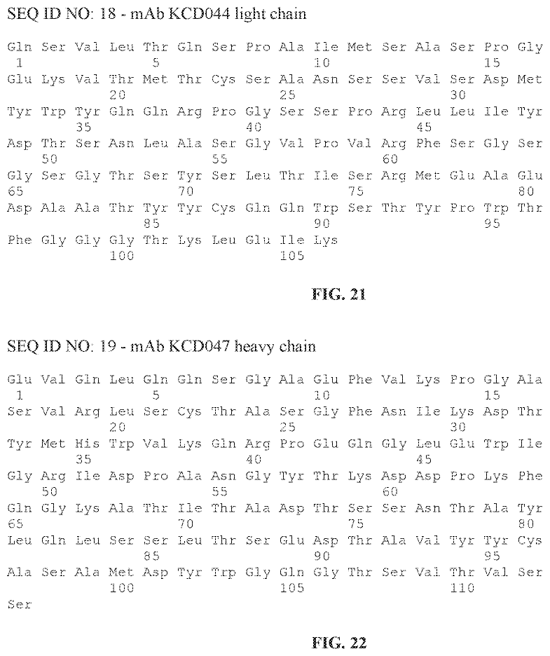

In some embodiments, the first and or second solution comprises a protein with the polynucleotide sequence of SEQ ID NO: 19. FIG. 22 shows some embodiments of a polynucleotide sequence of a mAb KCD047 heavy chain polynucleotide. (SEQ ID NO: 19).

In some embodiments, the first and or second solution comprises a protein with the polynucleotide sequence of SEQ ID NO: 20. FIG. 23 shows some embodiments of a polynucleotide sequence of a mAb KCD047 light chain polynucleotide. (SEQ ID NO: 20).

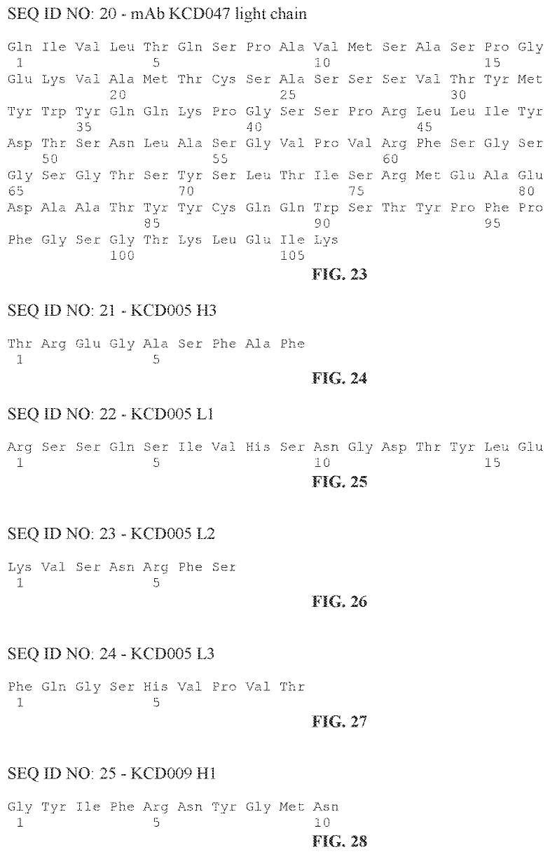

In some embodiments, the first and or second solution comprises a protein with the polynucleotide sequence of SEQ ID NO: 21. FIG. 24 shows some embodiments of a polynucleotide sequence of a KCD005 H3 polynucleotide. (SEQ ID NO: 21).

In some embodiments, the first and or second solution comprises a protein with the polynucleotide sequence of SEQ ID NO: 22. FIG. 25 shows some embodiments of a polynucleotide sequence of a KCD005 L1 polynucleotide. (SEQ ID NO: 22).

In some embodiments, the first and or second solution comprises a protein with the polynucleotide sequence of SEQ ID NO: 23. FIG. 26 shows some embodiments of a polynucleotide sequence of a KCD005 L2 polynucleotide. (SEQ ID NO: 23).

In some embodiments, the first and or second solution comprises a protein with the polynucleotide sequence of SEQ ID NO: 24. FIG. 27 shows some embodiments of a polynucleotide sequence of a KCD005 L3 polynucleotide. (SEQ ID NO: 24).

In some embodiments, the first and or second solution comprises a protein with the polynucleotide sequence of SEQ ID NO: 25. FIG. 28 shows some embodiments of a polynucleotide sequence of a KCD009 H1 polynucleotide. (SEQ ID NO: 25).

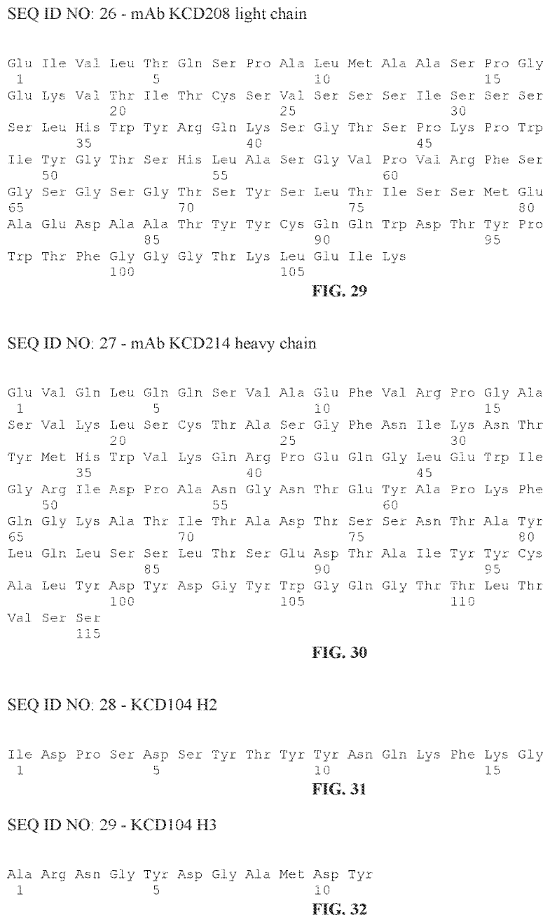

In some embodiments, the first and or second solution comprises a protein with the polynucleotide sequence of SEQ ID NO: 26. FIG. 29 shows some embodiments of a polynucleotide sequence of a mAb KCD208 light chain polynucleotide. (SEQ ID NO: 26).

In some embodiments, the first and or second solution comprises a protein with the polynucleotide sequence of SEQ ID NO: 27. FIG. 30 shows some embodiments of a polynucleotide sequence of a mAb KCD214 heavy chain polynucleotide. (SEQ ID NO: 27).

In some embodiments, the first and or second solution comprises a protein with the polynucleotide sequence of SEQ ID NO: 28. FIG. 31 shows some embodiments of a polynucleotide sequence of a KCD104 H2 polynucleotide. (SEQ ID NO. 28)

In some embodiments, the first and or second solution comprises a protein with the polynucleotide sequence of SEQ ID NO: 29. FIG. 32 shows some embodiments of a polynucleotide sequence of a KCD104 H3 polynucleotide. (SEQ ID NO: 29).

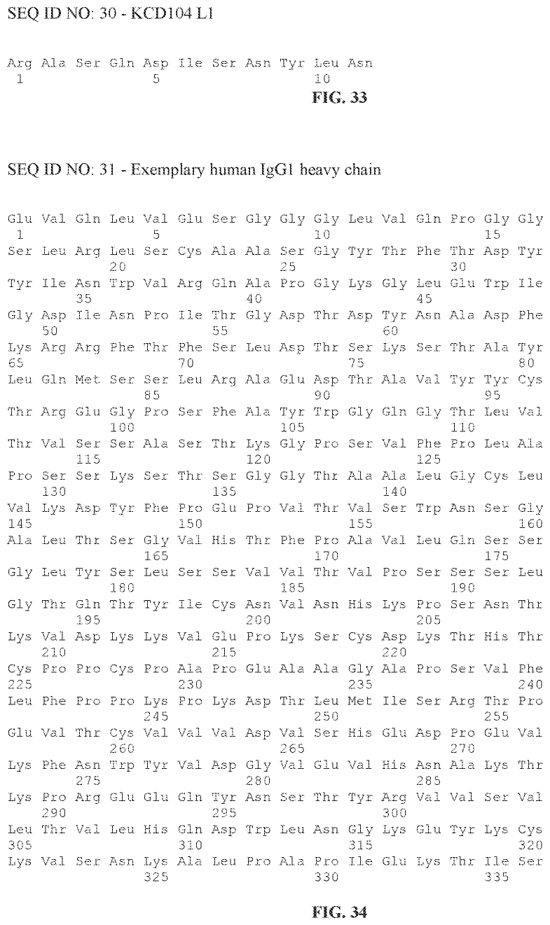

In some embodiments, the first and or second solution comprises a protein with the polynucleotide sequence of SEQ ID NO: 30. FIG. 33 shows some embodiments of a polynucleotide sequence of a KCD104 L1 polynucleotide. (SEQ ID NO: 30).

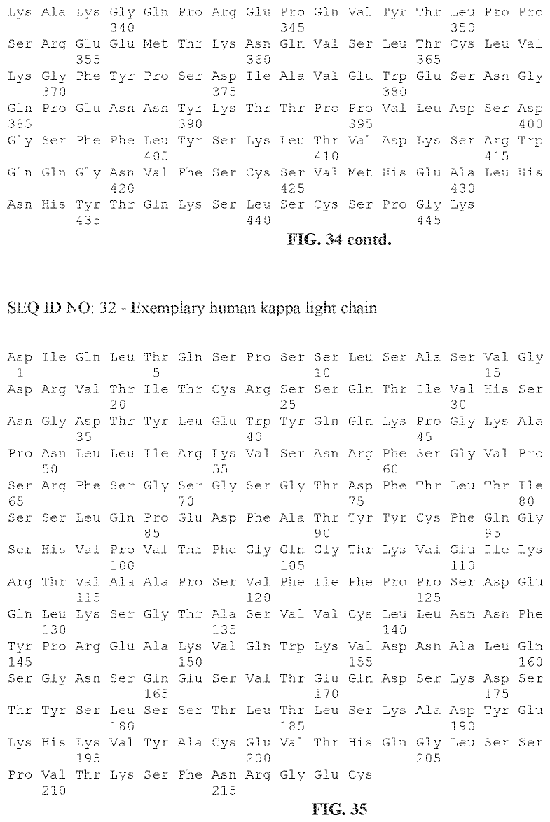

In some embodiments, the first and or second solution comprises a protein with the polynucleotide sequence of SEQ ID NO: 31. FIG. 34 shows some embodiments of a polynucleotide sequence of a Exemplary human IgG1 heavy chain polynucleotide. (SEQ ID NO: 31).

In some embodiments, the first and or second solution comprises a protein with the polynucleotide sequence of SEQ ID NO: 32. FIG. 35 shows some embodiments of a polynucleotide sequence of a Exemplary human kappa light chain polynucleotide. (SEQ ID NO: 32).

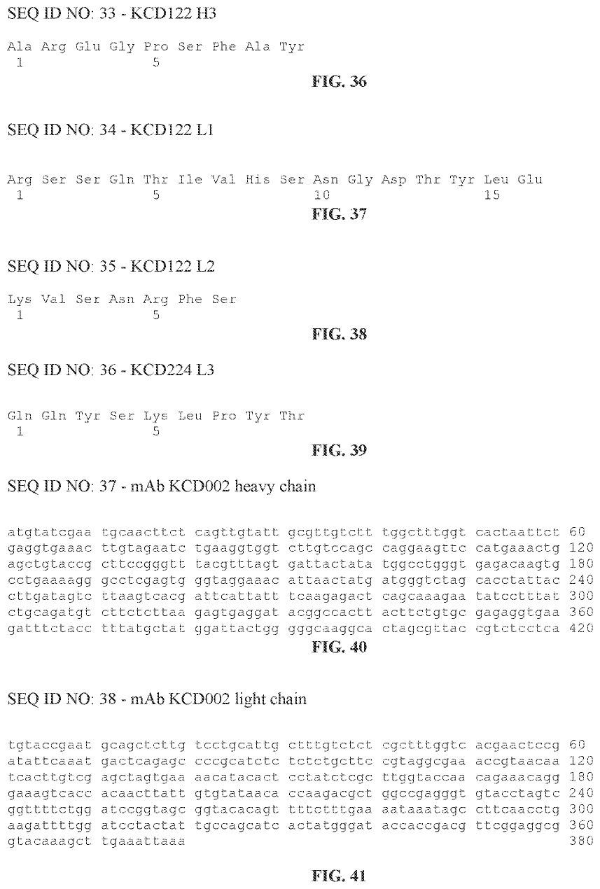

In some embodiments, the first and or second solution comprises a protein with the polynucleotide sequence of SEQ ID NO: 33. FIG. 36 shows some embodiments of a polynucleotide sequence of a KCD122 H3 polynucleotide. (SEQ ID NO: 33).

In some embodiments, the first and or second solution comprises a protein with the polynucleotide sequence of SEQ ID NO: 34. FIG. 37 shows some embodiments of a polynucleotide sequence of a KCD122 L1 polynucleotide. (SEQ ID NO: 34).

In some embodiments, the first and or second solution comprises a protein with the polynucleotide sequence of SEQ ID NO: 35. FIG. 38 shows some embodiments of a polynucleotide sequence of a KCD122 L2 polynucleotide. (SEQ ID NO: 35).

In some embodiments, the first and or second solution comprises a protein with the polynucleotide sequence of SEQ ID NO: 36. FIG. 39 shows some embodiments of a polynucleotide sequence of a KCD224 L3 polynucleotide. (SEQ ID NO: 36).

In some embodiments, the first and or second solution comprises a protein with the polynucleotide sequence of SEQ ID NO: 37. FIG. 40 shows some embodiments of a polynucleotide sequence of a mAb KCD002 heavy chain polynucleotide (SEQ ID NO: 37).

In some embodiments, the first and or second solution comprises a protein with the polynucleotide sequence of SEQ ID NO: 38. FIG. 41 shows some embodiments of a polynucleotide sequence of a mAb KCD002 light chain polynucleotide. (SEQ ID NO: 38).

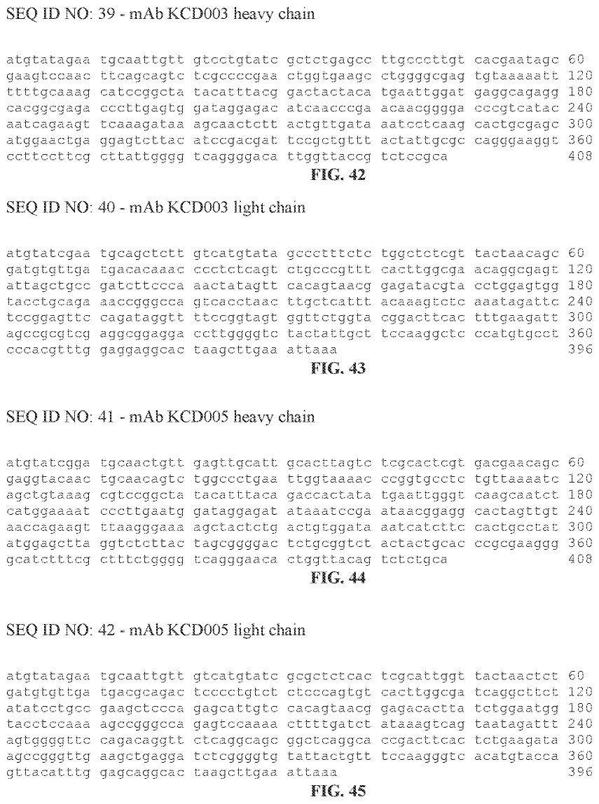

In some embodiments, the first and or second solution comprises a protein with the polynucleotide sequence of SEQ ID NO: 39. FIG. 42 shows some embodiments of a polynucleotide sequence of a mAb KCD003 heavy chain polynucleotide. (SEQ ID NO: 39).

In some embodiments, the first and or second solution comprises a protein with the polynucleotide sequence of SEQ ID NO: 40. FIG. 43 shows some embodiments of a polynucleotide sequence of a mAb KCD003 light chain polynucleotide. (SEQ ID NO: 40).

In some embodiments, the first and or second solution comprises a protein with the polynucleotide sequence of SEQ ID NO: 41. FIG. 44 shows some embodiments of a polynucleotide sequence of a mAb KCD005 heavy chain polynucleotide. (SEQ ID NO: 41).

In some embodiments, the first and or second solution comprises a protein with the polynucleotide sequence of SEQ ID NO: 42. FIG. 45 shows some embodiments of a polynucleotide sequence of a mAb KCD005 light chain polynucleotide. (SEQ ID NO: 42).

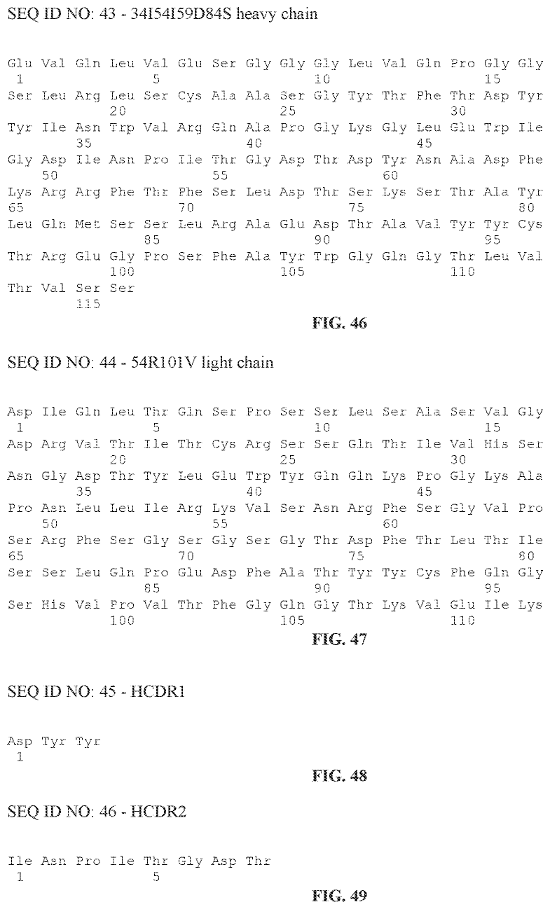

In some embodiments, the first and or second solution comprises a protein with the polynucleotide sequence of SEQ ID NO: 43. FIG. 46 shows some embodiments of a polynucleotide sequence of a 34I54I59D84S heavy chain polynucleotide. (SEQ ID NO: 43).

In some embodiments, the first and or second solution comprises a protein with the polynucleotide sequence of SEQ ID NO: 44. FIG. 47 shows some embodiments of a polynucleotide sequence of a 54R101V light chain p polynucleotide. (SEQ ID NO: 44).

In some embodiments, the first and or second solution comprises a protein with the polynucleotide sequence of SEQ ID NO: 45. FIG. 48 shows some embodiments of a polynucleotide sequence of a HCDR1 polynucleotide. (SEQ ID NO: 45).

In some embodiments, the first and or second solution comprises a protein with the polynucleotide sequence of SEQ ID NO: 46. FIG. 49 shows some embodiments of a polynucleotide sequence of a HCDR2 polynucleotide. (SEQ ID NO: 46).

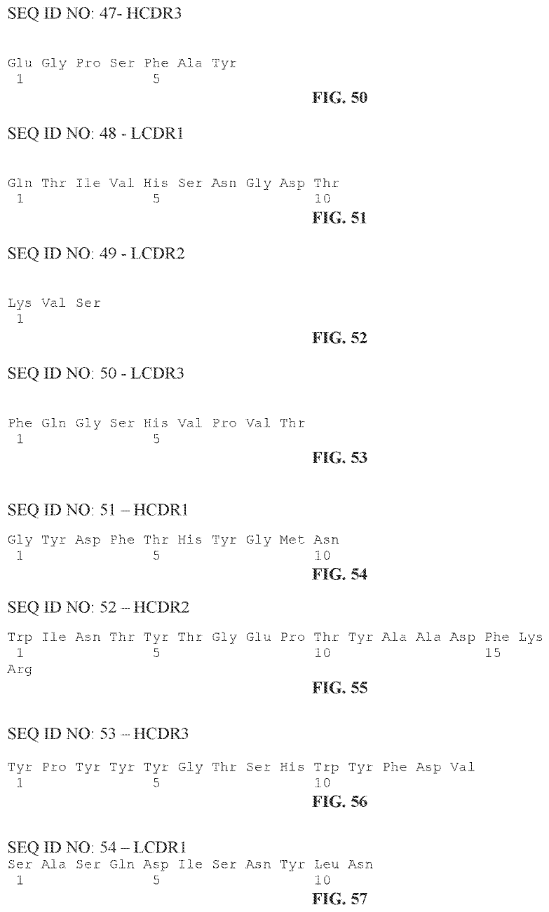

In some embodiments, the first and or second solution comprises a protein with the polynucleotide sequence of SEQ ID NO: 47. FIG. 50 shows some embodiments of a polynucleotide sequence of a HCDR3 polynucleotide. (SEQ ID NO: 47).

In some embodiments, the first and or second solution comprises a protein with the polynucleotide sequence of SEQ ID NO: 48. FIG. 51 shows some embodiments of a polynucleotide sequence of a LCDR1 polynucleotide. (SEQ ID NO: 48).

In some embodiments, the first and or second solution comprises a protein with the polynucleotide sequence of SEQ ID NO: 49. FIG. 52 shows some embodiments of a polynucleotide sequence of a LCDR2 polynucleotide. (SEQ ID NO: 49).

In some embodiments, the first and or second solution comprises a protein with the polynucleotide sequence of SEQ ID NO: 50. FIG. 53 shows some embodiments of a polynucleotide sequence of a LCDR3 polynucleotide. (SEQ ID NO: 50).

In some embodiments, the first and or second solution comprises a protein with the polynucleotide sequence of SEQ ID NO: 51. FIG. 54 shows some embodiments of a polynucleotide sequence of a HCDR1 polynucleotide. (SEQ ID NO: 51).

In some embodiments, the first and or second solution comprises a protein with the polynucleotide sequence of SEQ ID NO: 52. FIG. 55 shows some embodiments of a polynucleotide sequence of a HCDR2 polynucleotide. (SEQ ID NO: 52).

In some embodiments, the first and or second solution comprises a protein with the polynucleotide sequence of SEQ ID NO: 53. FIG. 56 shows some embodiments of a polynucleotide sequence of a HCDR3 polynucleotide. (SEQ ID NO: 53).

In some embodiments, the first and or second solution comprises a protein with the polynucleotide sequence of SEQ ID NO: 54. FIG. 57 shows some embodiments of a polynucleotide sequence of a LCDR1 polynucleotide. (SEQ ID NO: 54).

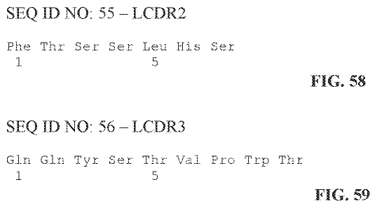

In some embodiments, the first and or second solution comprises a protein with the polynucleotide sequence of SEQ ID NO: 55. FIG. 58 shows some embodiments of a polynucleotide sequence of a LCDR2 polynucleotide. (SEQ ID NO: 55).

In some embodiments, the first and or second solution comprises a protein with the polynucleotide sequence of SEQ ID NO: 56. FIG. 59 shows some embodiments of a polynucleotide sequence of a LCDR1 polynucleotide. (SEQ ID NO: 56)

In some embodiments, one or more of the sequences and/or structures in FIG. 4-53 can be used herein for any one or more of the methods provided herein, including those in FIG. 1-3.

### EXAMPLES

### Example 1

The present example demonstrates a method of concentrating a viscous protein solution. A first solution comprising 510 g of Tarcocimab tedromer at a starting concentration of 13.4 g/L (based on antibody mass) formulated in 5 mM sodium phosphate, pH 6.5, 0.01% Polysorbate-20 was filled into a 1 L round-bottom flask and connected to a rotavapor. The pressure was decreased to 18 mbar over 1 h 30 min. The solution was then concentrated to 152 g under vacuum (16-18 mbar) at 35° C. during 1 h 15 min. 507 g of Tarcocimab tedromer at a starting concentration of 13.4 g/L (based on antibody mass) formulated in 5 mM sodium phosphate, pH 6.5, 0.01% Polysorbate-20 was added into the 1 L round-bottom flask and connected to a rotavapor. The pressure was decreased to 18 mbar over 29 min. The solution was concentrated to 133 g under vacuum (16-18 mbar) at 35° C. during 2 h 09 min. The solution was transferred into a 250 ml Nalgene bottle and the 1 L round-bottom flask rinsed twice with 5 ml of Milli-Q water. The rinse-water was added into the 250 ml Nalgene bottle, affording 259 g of Tarcocimab tedromer at a final concentration of 48.5 g/L (based on antibody mass) (95% yield).

### Example 2

The present example demonstrates a method of concentrating a viscous protein solution. A first solution comprising 1800 g of Tarcocimab tedromer at a starting concentration of 19.9 g/L (based on antibody mass) formulated in 5 mM sodium phosphate, pH 6.5, 0.01% Polysorbate-20 was filled into a 10 L glass vessel. The pressure was rapidly decreased to 100 mbar, and then gradually decreased to 3 mbar over 1 h 41 min. The solution was concentrated to 830 g under vacuum (2-7 mbar) at 11-14° C. during 8 h 39 min. The solution was filtered over a 0.2 μm filter, affording 328 g of Tarcocimab tedromer at a final concentration of 49.0 g/L (based on antibody mass) and a viscosity of 1277 mPa.s (45% yield).

### Example 3

The present example demonstrates a method of concentrating a viscous protein solution. A first solution comprising 1.74 kg of an antibody biopolymer conjugate (anti IL-6 and anti VEGF bispecific antibody) at a starting concentration of 19.8 g/L (based on antibody mass) formulated in 5 mM sodium acetate, pH 5.0, 0.01% Polysorbate-20 was filled into a 4 L glass vessel. The pressure was rapidly decreased to 100 mbar, and then gradually decreased to 19 mbar over 1 h 22 min. The solution was concentrated to 0.56 kg under vacuum (18-22 mbar) at 18-19° C. during 13 h 30 min. The solution was filtered over a 0.2 um filter, affording 0.55 kg of antibody biopolymer conjugate at a final concentration of 53.2 g/L (based on antibody mass) and a viscosity of 1056 mPa.s (85% yield).

### Example 4

The present example demonstrates a method of concentrating a viscous protein solution. A first solution comprising 10.166 kg of Tarcocimab tedromer at a starting concentration of 20.0 g/L (based on antibody mass) formulated in 5 mM sodium phosphate, pH 6.5, 0.01% Polysorbate-20 was filled into a 20 L stainless steel vessel. The pressure was rapidly decreased to 100 mbar, and then gradually decreased to 17 mbar over 1 h 23 min. The solution was concentrated to 3.60 L under vacuum (16-19 mbar) at 13-19° C. during 43 h 38 min. The solution was filtered over a 0.2 pm filter, affording 3.66 kg of Tarcocimab tedromer at a final concentration of 46.9 g/L (based on antibody mass) and a viscosity of 1812 mPa.s (84% yield).

### Example 5

The present example demonstrates a method of concentrating a viscous protein solution. A first solution comprising 111.0 L of Tarcocimab tedromer at a starting concentration of 19.9 g/L (based on antibody mass) formulated in 50 mM sodium acetate, pH 4.4, 0.01% Polysorbate-20 was filled into a 150 L stainless steel vessel. The pressure was rapidly decreased to 150 mbar, and then gradually decreased to 13 mbar over 1 h 50 min. The solution was concentrated to 41.4 L under vacuum (13 mbar) at 15° C. during 51 h 20 min. The solution was filtered over a 0.2 um filter, affording 31.9 kg of Tarcocimab tedromer at a final concentration of 51.9 g/L (based on antibody mass) and a viscosity of 315 mPa.s (70% yield).

Embodiments provided herein are further described in the following enumerated arrangements.
1. A method of concentrating a viscous protein solution, the method comprising: providing a first solution comprising protein; and reducing a volume of the first solution via evaporative concentration, wherein a vacuum is applied during evaporative concentration of at least below 100 mbar, to thereby produce a second solution, wherein the second solution has a viscosity between 400 to 4000 mPa.s or cP.
2. A method of concentrating a viscous protein solution, the method comprising: providing a first solution comprising protein; and reducing a volume of the first solution via evaporative concentration, wherein a vacuum is applied during evaporative concentration of at least below 100 mbar, to thereby produce a second solution, wherein the second solution has a viscosity between 250 to 4000 mPa.s or cP.
3. A method of reducing a volume of a therapeutic formulation, the method comprising: providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution; wherein the second solution has a viscosity between 400 to 4000 mPa.s or cP.
4. A method of reducing a volume of a therapeutic formulation, the method comprising: providing a first solution, the first solution comprising a conjugated polymer, wherein the first solution is at a first volume; and applying a vacuum of at least below 100 mbar to the first solution and reducing the first volume to a second volume by evaporative concentration to thereby produce a second solution; wherein the second solution has a viscosity between 250 to 4000 mPa.s or cP.
5. A method of reducing a volume of a therapeutic formulation, the method comprising: providing a first solution, the first solution comprising at least 0.001% polysorbate and a conjugate, wherein the conjugate comprises a polymer conjugated to an antibody or a small-molecule drug or both, optionally, wherein the first solution further comprises unconjugated antibody, wherein the first solution is at a first volume; and applying a vacuum of at least below 100 mbar to the first solution to reduce the first volume to a second volume by evaporative concentration to thereby produce a second solution, wherein the second solution has a viscosity between 400 to 4000 mPa.s or cP, wherein evaporative concentration is carried out at less than 40° C., and wherein the vacuum of at least below 100 mbar is established over at least 15 minutes to reduce foaming
6. A method of reducing a volume of a therapeutic formulation, the method comprising: providing a first solution, the first solution comprising at least 0.001% polysorbate and a conjugate, wherein the conjugate comprises a polymer conjugated to an antibody or a small-molecule drug or both, optionally, wherein the first solution further comprises unconjugated antibody, wherein the first solution is at a first volume; and applying a vacuum of at least below 100 mbar to the first solution to reduce the first volume to a second volume by evaporative concentration to thereby produce a second solution, wherein the second solution has a viscosity between 250 to 4000 mPa.s or cP, wherein evaporative concentration is carried out at less than 40° C., and wherein the vacuum of at least below 100 mbar is established over at least 15 minutes to reduce foaming.
7. The method of any of the preceding arrangements, further comprising agitation of the first and/or second solution.
8. The method of any of the preceding arrangements, wherein the first and/or second solution are agitated via stirring.
9. The method of any of the preceding arrangements, wherein the solution is stirred for a period of time lasting at least about 15 minutes.
10. The method of any of the preceding arrangements, wherein the solution is stirred for a period of time lasting between about 30 minutes and 6 hours.
11. The method of any of the preceding arrangements, wherein the solution is stirred for a period of time lasting between about 45 minutes and 3 hours.
12. The method of any of the preceding arrangements, wherein the solution is stirred for a period of time lasting at least about 1.5 hours.
13. The method of any of the preceding arrangements, wherein the solution is stirred for a period of time lasting at between about 24 and 96 hours.
14. The method of any of the preceding arrangements, wherein the solution is stirred for a period of time lasting at between about 48 and 72 hours.
15. The method of any of the preceding arrangements, wherein the solution is stirred for a period of time lasting about 48 hours.
16. The method of any of the preceding arrangements, wherein the solution is stirred for a period of time lasting about 60 hours.
17. The method of any of the preceding arrangements, wherein the solution is stirred for a period of time lasting about 72 hours.
18. The method of any of the preceding arrangements, wherein the solution is stirred at up to about 100 RPM.
19. The method of any of the preceding arrangements, wherein the solution is stirred at up to about 25 RPM.
20. The method of any of the preceding arrangements, wherein the solution is stirred at about 15 RPM.
21. The method of any of the preceding arrangements, wherein the solution is stirred continuously.
22. The method of any of the preceding arrangements, wherein the solution is stirred intermittently.
23. The method of any of the preceding arrangements, wherein the solution is stirred to homogeneity.
24. The method of any of the preceding arrangements, wherein the first solution reduced by evaporative concentration is reduced by between about 20% and 80%.
25. The method of any of the preceding arrangements, wherein the first solution reduced by evaporative concentration is reduced by between about 30% and 80%.
26. The method of any of the preceding arrangements, wherein the first solution reduced by evaporative concentration is reduced by between about 40% and 70%.
27. The method of any of the preceding arrangements, wherein the first solution reduced by evaporative concentration is reduced by up to about 60%.
28. The method of any of the preceding arrangements, wherein the first solution reduced by evaporative concentration is reduced by up to about 2.5-fold.
29. The method of any of the preceding arrangements, wherein the vacuum is increased to at least below 100 mbar over a period of time lasting at least about 15 minutes.
30. The method of any of the preceding arrangements, wherein the vacuum is increased to at least below 100 mbar over a period of time lasting between about 30 minutes and 6 hours.
31. The method of any of the preceding arrangements, wherein the vacuum is increased to at least below 100 mbar over a period of time lasting between about 45 minutes and 3 hours.
32. The method of any of the preceding arrangements, wherein the vacuum is increased to at least below 100 mbar over a period of time lasting at least about 1.5 hours.
33. The method of any of the preceding arrangements, wherein the vacuum applied to the first solution for up to about 96 hours.
34. The method of any of the preceding arrangements, wherein the vacuum applied to the first solution for between about 48 and 72 hours.
35. The method of any of the preceding arrangements, wherein the vacuum applied to the first solution for about 48 hours.
36. The method of any of the preceding arrangements, wherein the vacuum applied to the first solution for about 60 hours.
37. The method of any of the preceding arrangements, wherein the vacuum applied to the first solution for about 72 hours.
38. The method of any of the preceding arrangements, wherein the evaporative concentration process is monitored on-line.
39. The method of any of the preceding arrangements, wherein the concentration measurement comprises using ultraviolet light.
40. The method of any of the preceding arrangements, wherein the concentration measurement is made using FlowVPE.
41. The method of any of the preceding arrangements, wherein the volume measurement comprises using one or more of: radar, conductivity, viscosity, or osmolality.
42. The method of any of the preceding arrangements, wherein the first solution further comprises a surfactant.
43. The method of any of the preceding arrangements, wherein the volume of the first solution is between about 25 L and 125 L.
44. The method of any of the preceding arrangements, wherein the volume of the first solution is between about 25 L and 100 L.
45. The method of any of the preceding arrangements, wherein the first solution comprises between about 0.001% to 0.2% surfactant.
46. The method of any of the preceding arrangements, wherein the first solution comprises about 0.01% surfactant.
47. The method of any of the preceding arrangements, wherein the surfactant comprises fatty acid esters, sorbitol, and/or sorbitol derivatives.
48. The method of any of the preceding arrangements, wherein the surfactant comprises polysorbate.
49. The method of any of the preceding arrangements, wherein the polysorbate comprises Polysorbate-20.
50. The method of any of the preceding arrangements, wherein the polysorbate comprises Polysorbate-80.
51. The method of any of the preceding arrangements, wherein the first solution comprises polysorbate; and wherein said first solution is concentrated such that the concentration of polysorbate in the second solution is between 1-fold and 5-fold higher.
52. The method of any of the preceding arrangements, wherein the second solution comprises about 0.025% surfactant.
53. The method of any of the preceding arrangements, wherein the second solution has a viscosity of 250 to 2000 mPa.s or cP.
54. The method of any of the preceding arrangements, wherein the second solution has a viscosity of 750 to 2000 mPa.s or cP.
55. The method of any of the preceding arrangements, wherein the second solution has a viscosity of about 1000 mPa.s or cP.
56. The method of any of the preceding arrangements, wherein the second solution has a viscosity of about 315 mPa.s or cP.
57. The method of any of the preceding arrangements, wherein the evaporative concentration is carried out at vacuum below at least 100 mbar.
58. The method of any of the preceding arrangements, wherein the evaporative concentration is carried out at vacuum below at least 50 mbar.
59. The method of any of the preceding arrangements, wherein the vacuum applied is at least below 30 mbar.
60. The method of any of the preceding arrangements, wherein the evaporative concentration is carried out at vacuum of about 17 mbar.
61. The method of any of the preceding arrangements, wherein the vacuum below 100 mbar is established over a period of up to about 30 minutes.
62. The method of any of the preceding arrangements, wherein the pressure is reduced from atmospheric pressure to about 17 mbar at a rate of about 90 mbar/min.
63. The method of any of the preceding arrangements, wherein the pressure is reduced from below about 100 mbar to the target pressure at a rate of about 1 mbar/min.
64. The method of any of the preceding arrangements, wherein evaporative concentration is carried out at a temperature between about 40 degrees C. and freezing.
65. The method of any of the preceding arrangements, wherein evaporative concentration is carried out at a temperature between about 25 degrees C. and freezing.
66. The method of any of the preceding arrangements, wherein evaporative concentration is carried out at temperature at about 15 degrees C.
67. The method of any of the preceding arrangements, wherein evaporative concentration is carried out at a temperature between about 5 degrees C. and freezing.
68. The method of any of the preceding arrangements, wherein the concentration of the protein in the second solution is between 40 mg/mL and 60 mg/mL
69. The method of any of the preceding arrangements, wherein the concentration of the protein in the second solution is between 45.0 mg/mL and 52.5 mg/mL.
70. The method of any of the preceding arrangements, wherein the concentration of the protein in the second solution is about 47.5 mg/mL.
71. The method of any of the preceding arrangements, wherein the concentration of the protein in the second solution is about 50.0 mg/mL.
72. The method of any of the preceding arrangements, further comprising venting the second volume.
73. The method of any of the preceding arrangements, further comprising filtering the concentrated viscous protein solution.

74. The method of any of the preceding arrangements, further comprising collecting quality control samples during the evaporative concentration process.
75. The method of any of the preceding arrangements, further comprising storage of the concentrated viscous protein solution.
76. The method of any of the preceding arrangements, wherein the therapeutic formulation is a pharmaceutical composition comprising an isolated antagonistic antibody, and a pharmaceutically acceptable carrier.
77. The method of any of the preceding arrangements, wherein the therapeutic formulation comprises a formulation for the treatment or prophylaxis of an ocular disorder.
78. The method of any of the preceding arrangements, wherein the antibody is a conjugated antibody.
79. The method of any of the preceding arrangements, wherein the conjugated antibody is an isolated antagonist antibody comprising: a CDRH1 that is a CDRH1 in SEQ ID NO: 28; a CDRH1 that is a CDRH1 in SEQ ID NO: 29; a CDRH1 that is a CDRH1 in SEQ ID NO: 30; a CDRH1 that is a CDRH1 in SEQ ID NO: 33; a CDRH1 that is a CDRH1in SEQ ID NO: 34; a CDRH1 that is a CDRH1 in SEQ ID NO: 35; at least one of the following mutations (EU numbering): L234A, L235A, and G237A; and at least one of the following mutations (EU numbering): Q347C or L443C.
80. The method of the preceding arrangements, wherein the conjugated antibody comprises an isolated antagonist IL-6 antibody comprising: a heavy chain amino acid variable region that comprises a heavy chain that has a sequence of at least one of SEQ ID NOs: 2-17, 21, 22, 36-42; and a light chain amino acid variable region that comprises the light chain that has a sequence of at least one of SEQ ID NOs: 18-20, 23-25.
81. The method of any of the preceding arrangements, wherein the conjugated antibody comprises an anti-VEGF antibody.
82. The method of any of the preceding arrangements, wherein the anti-VEGF antibody heavy chain comprises a CDRH1 that is a CDRH1 in SEQ ID NO.: 51; a CDRH2 that is a CDRH2 in SEQ ID NO.: 52; and a CDRH3 that is a CDRH3 in SEQ ID NO: 53.
83. The method of any of the preceding arrangements, wherein the anti-VEGF antibody heavy chain comprises a CDRL1 that is a CDRL1 in SEQ ID NO.: 54; a CDRL2 that is a CDRL2 in SEQ ID NO: 55; and a CDRL3 that is a CDRL3 in SEQ ID NO: 56.
84. The method of any of the preceding arrangements, wherein the anti-VEGF antibody comprises: a heavy chain, wherein the heavy chain comprises a CDRH1 that is a CDRH1 in SEQ ID NO.: 51; a CDRH2 that is a CDRH2 in SEQ ID NO.: 52; a CDRH3 that is a CDRH3 in SEQ ID NO: 53; a light chain; wherein the heavy chain comprises a CDRL1 that is a CDRL1 in SEQ ID NO.: 54; a CDRL2 that is a CDRL2 in SEQ ID NO.: 55; a CDRL3 that is a CDRL3 in SEQ ID NO: 56; and wherein the heavy chain constant domain of the anti-VEGF-A antibody has one or more mutations relative to the constant domain of human IgG1 to modulate effector function, wherein the mutations are to one or more of the following amino acid positions (EU numbering): E233X, L234X, L235X, G236X, G237X, A327X, A330X, and P331X wherein X is any natural or unnatural amino acid, wherein the mutations are selected from the group consisting of (EU numbering): E233P, L234V, L234A, L235A, G237A, A327G, A330S, and P331S.
85. The method of any of the preceding arrangements, wherein the conjugated antibody comprises an isolated antibody that specifically binds to complement factor D (CFD) and directly inhibits a proteolytic activity of CFD, wherein the antibody comprises: a heavy chain variable region (VH) comprising: a VH complementarity determining region 1 (CDRH1) that is the CDRH1 in SEQ ID NO: 43; a CDRH2 that is the CDRH2 in SEQ ID NO: 43; and a CDRH3 that is the CDRH3 in SEQ ID NO: 43; and a light chain variable region (VL) comprising: a VL complementarity determining region 1 (CDRL1) comprising H31, N33, G34 or E34 or F34 or S34, D35 or E35, T36 or S36 or V36, Y37, L38 or 138, and E39 (EU numbering); a CDRL2 comprising L51 or H51, 153 or V53, and K55 (EU numbering); and a CDRL3 comprising F94 or L94, Q95, G96, S97, V99 or N99 or Q99 or W99, P100, and P101 or V101 (EU numbering).
86. The method of any of the preceding arrangements, wherein the conjugated antibody comprises an isolated antagonistic antibody that binds to CFD, the antibody comprising: a VH comprising: a CDRH1 that is the CDRH1 in SEQ ID NO: 43; a CDRH2 that is the CDRH2 in SEQ ID NO: 43; a CDRH3 that is the CDRH3 in SEQ ID NO: 43; a VL comprising: a CDRL1 that is the CDRL1 in SEQ ID NO: 44; a CDRL2 that is the CDRL2 in SEQ ID NO: 44; a CDRL3 that is the CDRL3 in SEQ ID NO: 44; at least one of the following mutations (EU numbering): L234A, L235A, and G237A; and at least one of the following mutations (EU numbering): Q347C or L443C.
87. The method of any one of the preceding arrangements, wherein the conjugate has the following structure:

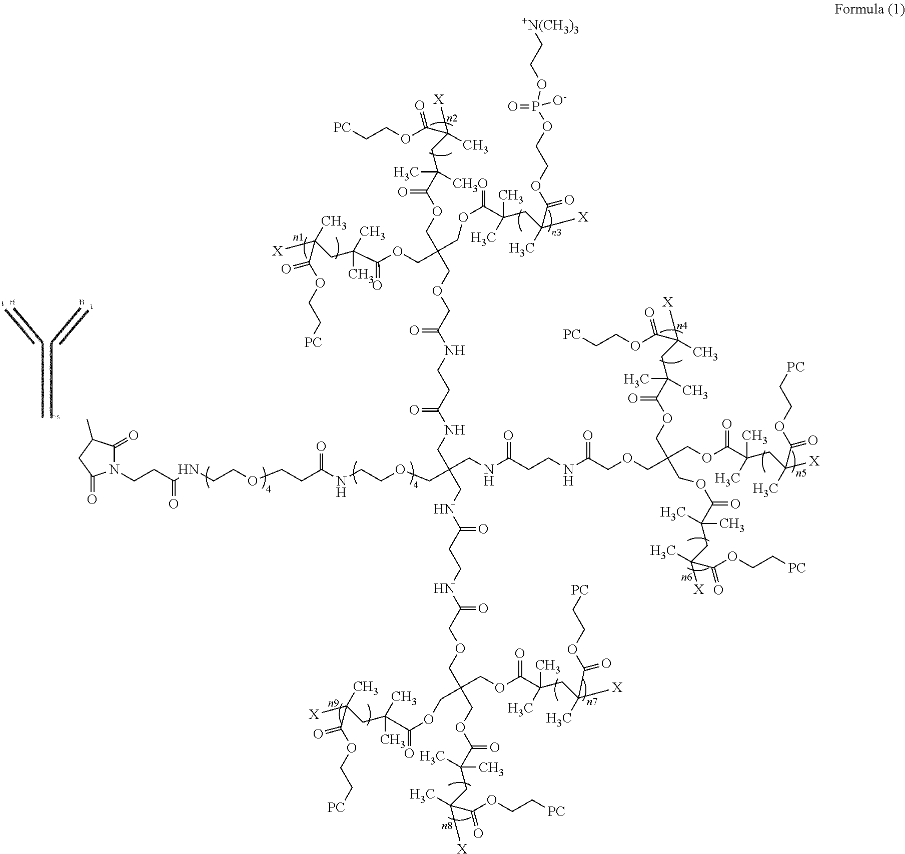

wherein: each heavy chain of the antibody is denoted by the letter H, and each light chain of the antibody is denoted by the letter L; the polymer is bonded to the antibody through the sulfhydryl of C443 (EU numbering), which bond is depicted on one of the heavy chains;

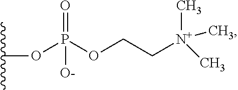 [SMILES: *OP(=O)([O-])OCC[N+](C)(C)C]

PC is where the curvy line indicates the point of attachment to the rest of the polymer, where X=a) OR where R═H, methyl, ethyl, propyl, isopropyl, b) H, or c) any halide, including Br; and n1, n2, n3, n4, n5, n6, n7, n8 and n9 are the same or different such that the sum of n1, n2, n3, n4, n5, n6, n6, n7, n8 and n9 is 2500 plus or minus 15%.
88. The method of any of the preceding arrangements, wherein the protein comprises a fusion protein.
89. The method of any of the preceding arrangements, wherein the protein is a fusion protein comprising a vascular endothelial growth factor (hereinafter “VGEF”) antagonist linked to a platelet-derived growth factor (herein after “PDGFR”) extracellular trap segment, wherein the PDGFR extracellular trap segment comprises domains D1-D3 of PDGFR-β.
90. The method of any of the preceding arrangements, wherein the antibody is conjugated to a polymer.
91. The method of any of the preceding arrangements, wherein the fusion protein is conjugated to a polymer.
92. The method of any of the preceding arrangements, wherein the polymer is covalently bonded to the antibody at a cysteine outside a variable region of the antibody wherein said cysteine has been added via recombinant technology.
93. The method of any of the preceding arrangements, wherein the polymer is a biopolymer.
94. The method of any of the preceding arrangements, wherein the biopolymer comprises an aflibercept biopolymer.
95. The method of any of the preceding arrangements, wherein the polymer comprises a phosphorylcholine polymer.
96. The method of any of the preceding arrangements, wherein the phosphorylcholine containing polymer comprises 2-(methacryloyloxyethyl)-2′-(trimethylammonium)ethyl phosphate (MPC) monomers as set forth below:

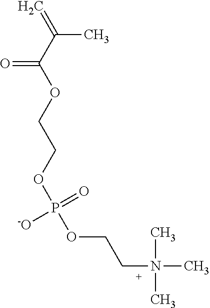

97. The method of any of the preceding arrangements, wherein the polymer has three or more arms or is synthesized with an initiator comprising 3 or more polymer initiation sites.
98. The method of any of the preceding arrangements, wherein the polymer has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.
99. The method of any of the preceding arrangements, wherein the polymer has 2, 3, 6, or 9 arms or is synthesized with an initiator comprising 2, 3, 6, or 9 polymer initiation sites.
100. The method of any of the preceding arrangements, wherein the polymer has 9 arms or is synthesized with an initiator comprising 9 polymer initiation sites.
101. The method of any of the preceding arrangements, wherein the polymer has a molecular weight between about 300,000 and about 1,750,000 Da as measured by SEC-MALS.
102. The method of any of the preceding arrangements, wherein the polymer has a molecular weight between about 500,000 and about 1,000,000 Da.
103. The method of any of the preceding arrangements, wherein the polymer has a molecular weight between about 750,000 to about 850,000 Da.
104. The method of any of the preceding arrangements, wherein the polymer comprises a zwitterionic monomer, wherein the zwitterionic monomer is selected from the group consisting of HEMA-phosphorylcholine, PEG, biocompatible fatty acids and derivatives thereof, Hydroxy Alkyl Starch (HAS), Hydroxy Ethyl Starch (HES), Poly Ethylene Glycol (PEG), Poly (Glyx-Sery) (HAP), Hyaluronic Acid (HA), Heparosan polymers (HEP), Fleximers, Dextran, Poly-sialic acids (PSA), FC domains, Transferrin, 25 Albumin, Elastin Like Peptides (ELP), CTP peptides, and FCRn binding peptides.

1. A method of concentrating a solution, the method comprising: providing a first solution comprising protein; and
applying a vacuum of at least below 100 mbar to the first solution and reducing the first volume to a second volume by evaporative concentration to thereby produce a second solution; wherein the second solution has a viscosity between 250 to 4000 mPa.s or cP.

2-24. (canceled)

25. The method of claim 1, wherein the first solution comprises a conjugated polymer.

26. The method of claim 25, wherein the polymer is conjugated to the protein or a small-molecule drug or both.

27. The method of claim 26, wherein the first solution comprises at least 0.001% polysorbate.

28. The method of claim 26, wherein evaporative concentration is carried out at 40° C. or less.

29. The method of claim 26, wherein the vacuum of at least below 100 mbar is established over at least 15 minutes to reduce foaming.

30. The method of claim 26, wherein 60 to 90% of the protein is conjugated to the polymer.

31. The method of claim 26, wherein the polymer comprises a phosphorylcholine polymer, wherein the phosphorylcholine containing polymer is formed by 2-(methacryloyloxyethyl)-2′-(trimethylammonium)ethyl phosphate (MPC) monomers as set forth below:

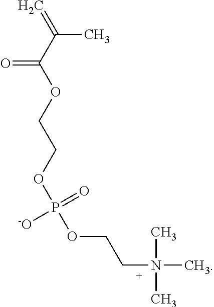

32. The method of claim 26, wherein the first solution reduced by evaporative concentration is reduced by between about 20% and 80%.

33. The method of claim 26, wherein the volume of the first solution is between about 25 L and 125 L.

34. The method of claim 26, wherein the second solution has a viscosity of 250 to 2000 mPa.s or cP.

35. The method of claim 26, wherein the concentration of the protein in the second solution is between 40 mg/mL and 60 mg/mL.

36. The method of claim 26, wherein the protein comprises a fusion protein. 37 (New) The method of claim 36, wherein the fusion protein comprises a vascular endothelial growth factor (hereinafter “VEGF”) antagonist linked to a platelet-derived growth factor (herein after “PDGFR”) extracellular trap segment, wherein the PDGFR extracellular trap segment comprises domains D1-D3 of PDGFR-β.

38. The method of claim 26, wherein the first solution is a pharmaceutical composition comprising an isolated antagonistic antibody, and a pharmaceutically acceptable carrier.

39. The method of claim 26, wherein the protein comprises an antibody.

40. The method of claim 39, wherein the conjugated polymer has the following structure: 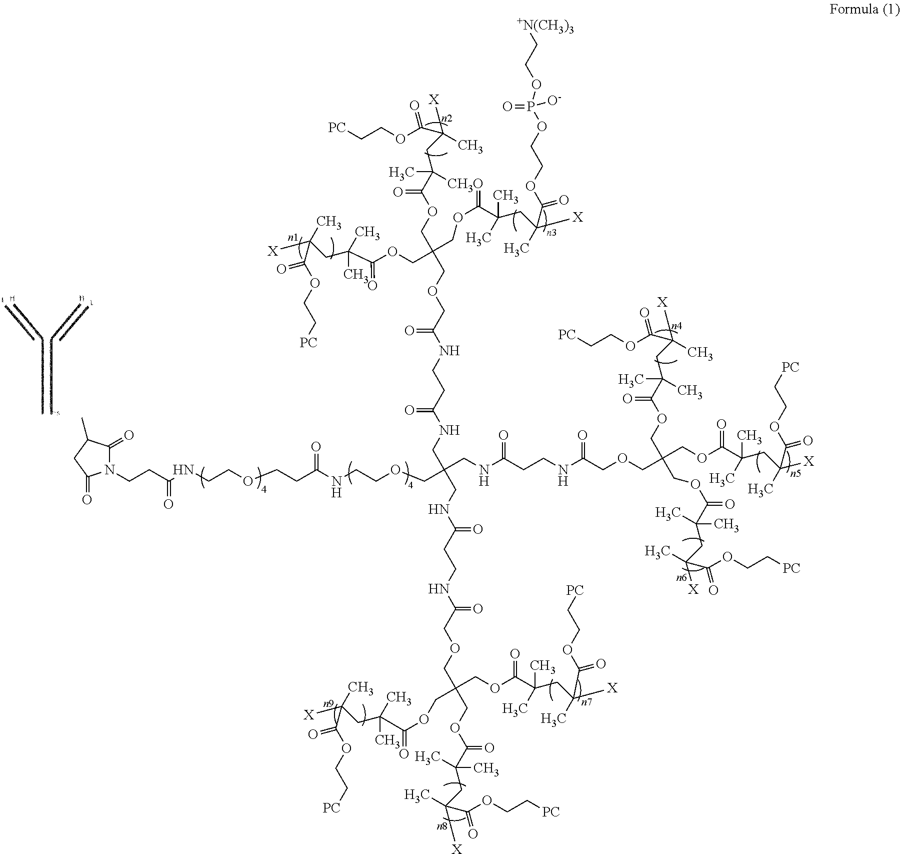
wherein:
the antibody comprises heavy chains denoted by the letter H, and light chains denoted by the letter L;
the polymer is bonded to the antibody through the sulfhydryl of C443 (EU numbering), which bond is depicted on one of the heavy chains;
PC is 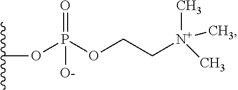 [SMILES: *OP(=O)([O-])OCC[N+](C)(C)C]
where the curvy line indicates the point of attachment to the rest of the polymer, where X is a) OR where R is H, methyl, ethyl, propyl, isopropyl, b) H, or c) a halogen; and
n1, n2, n3, n4, n5, n6, n7, n8 and n9 are the same or different such that the sum of n1, n2, n3, n4, n5, n6, n7, n8 and n9 is 2500 plus or minus 15%.

41. The method of claim 39, wherein the first solution further comprises unconjugated antibody.

42. The method of claim 39, wherein the antibody is a conjugated antibody, wherein the conjugated antibody comprises an anti-VEGF antibody, wherein the antibody comprises:
a heavy chain comprising:
a CDRH1 that is a CDRH1 in SEQ ID NO: 51;
a CDRH2 that is a CDRH2 in SEQ ID NO: 52;
a CDRH3 that is a CDRH3 in SEQ ID NO: 53; and
a heavy chain constant domain;
a light chain comprising:
a CDRL1 that is a CDRL1 in SEQ ID NO: 54;
a CDRL2 that is a CDRL2 in SEQ ID NO: 55; and
a CDRL3 that is a CDRL3 in SEQ ID NO: 56; and wherein
the heavy chain constant domain of the anti-VEGF-A antibody has one or more mutations relative to the constant domain of human IgG1 to modulate effector function, wherein the mutations are to one or more of the following amino acid positions (EU numbering): E233X, L234X, L235X, G236X, G237X, A327X, A330X, and P331X wherein X is any amino acid, wherein the mutations are selected from the group consisting of (EU numbering): E233P, L234V, L234A, L235A, G237A, A327G, A330S, and P331S.

43. The method of claim 39, wherein the antibody is a conjugated antibody, wherein the antibody comprises an anti IL-6 and anti-VEGF bispecific antibody.

44. The method of claim 39, wherein the polymer is covalently bonded to the antibody at a cysteine outside a variable region of the antibody wherein said cysteine has been added via recombinant technology.

45. A method of concentrating a first solution, the method comprising:
providing a first solution comprising protein; and
applying a vacuum of at least below 100 mbar to the first solution and reducing the first volume to a second volume by evaporative concentration to thereby produce a second solution;
wherein the second solution has a viscosity between 250 to 4000 mPa.s or cP,
wherein the first solution comprises a conjugated polymer,
wherein the polymer is conjugated to the protein or a small-molecule drug or both;
wherein the first solution comprises at least 0.001% polysorbate;
wherein evaporative concentration is carried out at less than 40° C.;
wherein the vacuum of at least below 100 mbar is established over at least 15 minutes to reduce foaming;
wherein 60 to 90% of the protein is conjugated to a polymer;
wherein the protein comprises an antibody or fusion protein.

46. The method of claim 45, wherein the conjugated polymer has the following structure: 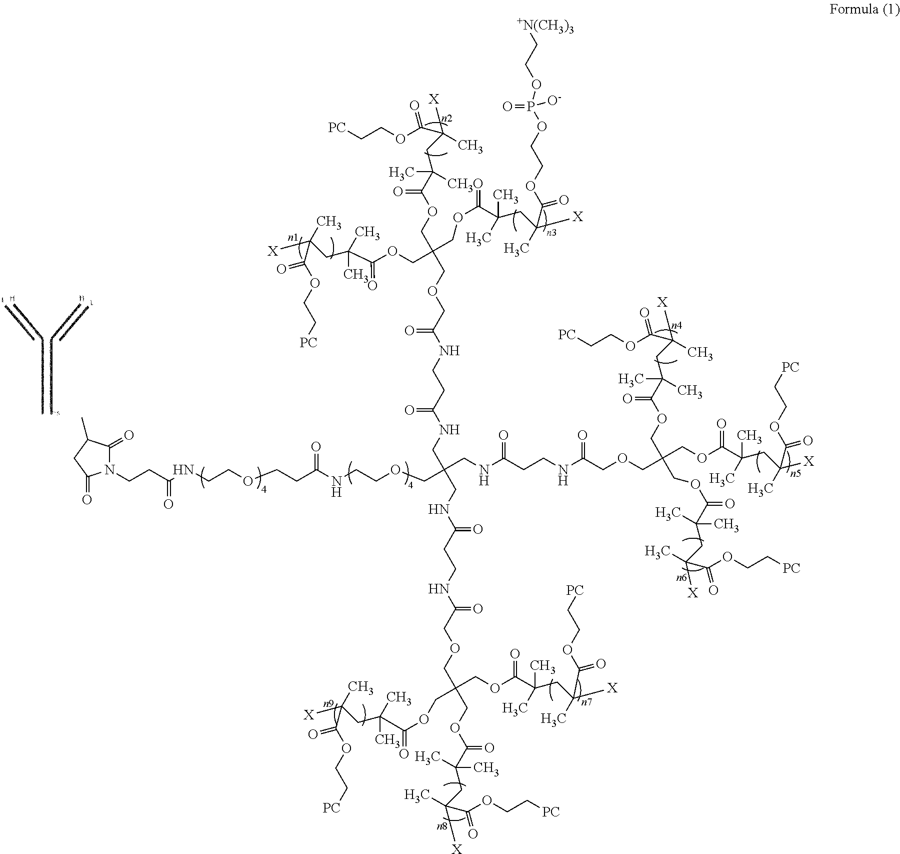
wherein:
the antibody comprises heavy chains denoted by the letter H, and light chains denoted by the letter L;
the polymer is bonded to the antibody through the sulfhydryl of C443 (EU numbering), which bond is depicted on one of the heavy chains;
PC is 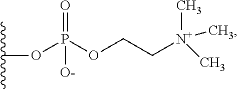 [SMILES: *OP(=O)([O-])OCC[N+](C)(C)C]
where the curvy line indicates the point of attachment to the rest of the polymer, where X is a) OR where Ris H, methyl, ethyl, propyl, isopropyl, b) H, or c) a halogen; and
n1, n2, n3, n4, n5, n6, n7, n8 and n9 are the same or different such that the sum of n1, n2,n3, n4, n5, n6, n7, n8 and n9 is 2500 plus or minus 15%.

47. The method of claim 45, wherein the first solution further comprises an unconjugated antibody.


SEQUENCE LISTING (56 sequences)

SEQ ID NO: 1 - protein, 228 aa, synthetic construct
  REGION 1..228: note = Mature form of human complement factor D
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 2 - protein, 116 aa, synthetic construct
  REGION 1..116: note = mAb KCD005 heavy chain
EVQLQQSGPELVKPGASVKISCKASGYTFTDHYMNWVKQSHGKSLEWIGDINPNNGGTSCNQKFKGKATLTVDKSSSTAYMELRSLTSGDSAVYYCTREGASFAFWGQGTLVTVSA

SEQ ID NO: 3 - protein, 112 aa, synthetic construct
  REGION 1..112: note = mAb KCD005 light chain
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPVTFGAGTKLELK

SEQ ID NO: 4 - protein, 10 aa, synthetic construct
  REGION 1..10: note = CDRH1
GYDFTHYGMN

SEQ ID NO: 5 - protein, 17 aa, synthetic construct
  REGION 1..17: note = CDRH2
WINTYTGEPTYAADFKR

SEQ ID NO: 6 - protein, 14 aa, synthetic construct
  REGION 1..14: note = CDRH3
YPYYYGTSHWYFDV

SEQ ID NO: 7 - protein, 11 aa, synthetic construct
  REGION 1..11: note = CDRL1
SASQDISNYLN

SEQ ID NO: 8 - protein, 7 aa, synthetic construct
  REGION 1..7: note = CDRL2
FTSSLHS

SEQ ID NO: 9 - protein, 114 aa, synthetic construct
  REGION 1..114: note = mAb KCD023 heavy chain
EVLLQQSGPELVKPGASVKIPCKASGYTFTDYNIDWVKQSHGKSLEWIGDINPNNGGINYNQKFKGKATLTVDKSSSTAYMELRSLTSEDTAVYYCGTGDYAYWGQGTLVTVSA

SEQ ID NO: 10 - protein, 108 aa, synthetic construct
  REGION 1..108: note = mAb KCD023 light chain
QIVLTQSPAFMSASPGEKVTLTCSASSSVSSSYLYWYQQKPGSSPKLWIYSTSNLASGVPGRFSGSGSGTSYSLTISSMEAEDAASYFCHQWTSYPPTFGAGTKLELK

SEQ ID NO: 11 - protein, 118 aa, synthetic construct
  REGION 1..118: note = mAb KCD036 heavy chain
EVQLQQSGPELVKPGASMKISCKASGYSFTGYTMTWVKQSHGKNLEWIGLINPYNGGTNYNQKFKGKATFTVDKSSSTAYMELLSLTSEDSAVYYCARRHYGSSWDYWGQGTTLTVSS

SEQ ID NO: 12 - protein, 107 aa, synthetic construct
  REGION 1..107: note = mAb KCD036 light chain
DIKLTQSPSSMYASLGERVTITCKASQDINTYLSWFQQKPGKSPKTLIYRANRLVDGVPSRFSGSGSGQDYSLTISSLEYEEMGIYYCLQYDEFPYTFGGGTKLEIK

SEQ ID NO: 13 - protein, 113 aa, synthetic construct
  REGION 1..113: note = mAb KCD040 heavy chain
EVQLQQSGAELVKPGASVKLSCTASDFNIKDTYMHWVMQRPEQGLEWIGKIDPANGNTEFDPKFQGKATITADTSSNTAYLQLTSLTSEDTAVYYCTRAMDYWGQGTSVTVSS

SEQ ID NO: 14 - protein, 106 aa, synthetic construct
  REGION 1..106: note = mAb KCD040 light chain
QIVLTQSPAIMSASPGEKVTMTCSAGSSVSYMYWYQQKPGSSPRVLIYDTSNLASGVPVRFSGSGSGTSYSLTISRMEAEDAATYYCQQWSNYPYTFGGGTKLEIK

SEQ ID NO: 15 - protein, 113 aa, synthetic construct
  REGION 1..113: note = mAb KCD042 heavy chain
EVQLQQSGAELVKPGASVRLSCTASGFNIKHTYIHWVSQRPEQGLEWIGKIDPANGNTKYDPKFQGKATITADTSSNTAYLQLSSLTSEDTAVYYCVNAMEYWGQGTSVTVSS

SEQ ID NO: 16 - protein, 106 aa, synthetic construct
  REGION 1..106: note = mAb KCD042 light chain
QSVLTQSPAIMSASPGEKVTMTCSANSSVSDMYWFQQRPGSSPRLLIYDTSNLASGVPVRFSGSGSGTSYSLTISRMEAEDAATYYCQQWSTYPWTFGGGTKLEIK

SEQ ID NO: 17 - protein, 113 aa, synthetic construct
  REGION 1..113: note = mAb KCD044 heavy chain
EVQLQQSGAELVKPGASVRLSCTASGFNIKHTYMHWVSQRPERGLEWIGKIDPANGNTKYDPKFQGKATITADTSSNTVYLQLSSLTSEDTAVYYCLNAMEYWGQGTSVTVSS

SEQ ID NO: 18 - protein, 106 aa, synthetic construct
  REGION 1..106: note = mAb KCD044 light chain
QSVLTQSPAIMSASPGEKVTMTCSANSSVSDMYWYQQRPGSSPRLLIYDTSNLASGVPVRFSGSGSGTSYSLTISRMEAEDAATYYCQQWSTYPWTFGGGTKLEIK

SEQ ID NO: 19 - protein, 113 aa, synthetic construct
  REGION 1..113: note = mAb KCD047 heavy chain
EVQLQQSGAEFVKPGASVRLSCTASGFNIKDTYMHWVKQRPEQGLEWIGRIDPANGYTKDDPKFQGKATITADTSSNTAYLQLSSLTSEDTAVYYCASAMDYWGQGTSVTVSS

SEQ ID NO: 20 - protein, 106 aa, synthetic construct
  REGION 1..106: note = mAb KCD047 light chain
QIVLTQSPAVMSASPGEKVAMTCSASSSVTYMYWYQQKPGSSPRLLIYDTSNLASGVPVRFSGSGSGTSYSLTISRMEAEDAATYYCQQWSTYPFPFGSGTKLEIK

SEQ ID NO: 21 - protein, 9 aa, synthetic construct
  REGION 1..9: note = KCD005 H3
TREGASFAF

SEQ ID NO: 22 - protein, 16 aa, synthetic construct
  REGION 1..16: note = KCD005 L1
RSSQSIVHSNGDTYLE

SEQ ID NO: 23 - protein, 7 aa, synthetic construct
  REGION 1..7: note = KCD005 L2
KVSNRFS

SEQ ID NO: 24 - protein, 9 aa, synthetic construct
  REGION 1..9: note = KCD005 L3
FQGSHVPVT

SEQ ID NO: 25 - protein, 10 aa, synthetic construct
  REGION 1..10: note = KCD009 H1
GYIFRNYGMN

SEQ ID NO: 26 - protein, 108 aa, synthetic construct
  REGION 1..108: note = mAb KCD208 light chain
EIVLTQSPALMAASPGEKVTITCSVSSSISSSSLHWYRQKSGTSPKPWIYGTSHLASGVPVRFSGSGSGTSYSLTISSMEAEDAATYYCQQWDTYPWTFGGGTKLEIK

SEQ ID NO: 27 - protein, 115 aa, synthetic construct
  REGION 1..115: note = mAb KCD214 heavy chain
EVQLQQSVAEFVRPGASVKLSCTASGFNIKNTYMHWVKQRPEQGLEWIGRIDPANGNTEYAPKFQGKATITADTSSNTAYLQLSSLTSEDTAIYYCALYDYDGYWGQGTTLTVSS

SEQ ID NO: 28 - protein, 16 aa, synthetic construct
  REGION 1..16: note = KCD104 H2
IDPSDSYTYYNQKFKG

SEQ ID NO: 29 - protein, 11 aa, synthetic construct
  REGION 1..11: note = KCD104 H3
ARNGYDGAMDY

SEQ ID NO: 30 - protein, 11 aa, synthetic construct
  REGION 1..11: note = KCD104 L1
RASQDISNYLN

SEQ ID NO: 31 - protein, 446 aa, synthetic construct
  REGION 1..446: note = Exemplary human IgG1 heavy chain
EVQLVESGGGLVQPGGSLRLSCAASGYTFTDYYINWVRQAPGKGLEWIGDINPITGDTDYNADFKRRFTFSLDTSKSTAYLQMSSLRAEDTAVYYCTREGPSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 32 - protein, 219 aa, synthetic construct
  REGION 1..219: note = Exemplary human kappa light chain
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 9 aa, synthetic construct
  REGION 1..9: note = KCD122 H3
AREGPSFAY

SEQ ID NO: 34 - protein, 16 aa, synthetic construct
  REGION 1..16: note = KCD122 L1
RSSQTIVHSNGDTYLE

SEQ ID NO: 35 - protein, 7 aa, synthetic construct
  REGION 1..7: note = KCD122 L2
KVSNRFS

SEQ ID NO: 36 - protein, 9 aa, synthetic construct
  REGION 1..9: note = KCD224 L3
QQYSKLPYT

SEQ ID NO: 37 - DNA, 420 bp, synthetic construct
  misc_feature 1..420: note = mAb KCD002 heavy chain
  source 1..420: mol_type = other DNA
atgtatcgaatgcaacttctcagttgtattgcgttgtctttggctttggtcactaattctgaggtgaaacttgtagaatctgaaggtggtcttgtccagccaggaagttccatgaaactgagctgtaccgcttccgggtttacgtttagtgattactatatggcctgggtgagacaagtgcctgaaaagggcctcgagtgggtaggaaacattaactatgatgggtctagcacctattaccttgatagtcttaagtcacgattcattatttcaagagactcagcaaagaatatcctttatctgcagatgtcttctcttaagagtgaggatacggccacttacttctgtgcgagaggtgaagatttctacctttatgctatggattactgggggcaaggcactagcgttaccgtctcctca

SEQ ID NO: 38 - DNA, 380 bp, synthetic construct
  misc_feature 1..380: note = mAb KCD002 light chain
  source 1..380: mol_type = other DNA
tgtaccgaatgcagctcttgtcctgcattgctttgtctctcgctttggtcacgaactccgatattcaaatgactcagagccccgcatctctctctgcttccgtaggcgaaaccgtaacaatcacttgtcgagctagtgaaaacatacactcctatctcgcttggtaccaacagaaacagggaaagtcaccacaacttattgtgtataacaccaagacgctggccgagggtgtacctagtcggttttctggatccggtagcggtacacagttttctttgaaaataaatagccttcaacctgaagattttggatcctactattgccagcatcactatgggataccaccgacgttcggaggcggtacaaagcttgaaattaaa

SEQ ID NO: 39 - DNA, 408 bp, synthetic construct
  misc_feature 1..408: note = mAb KCD003 heavy chain
  source 1..408: mol_type = other DNA
atgtatagaatgcaattgttgtcctgtatcgctctgagccttgcccttgtcacgaatagcgaagtccaacttcagcagtctcgccccgaactggtgaagcctggggcgagtgtaaaaattttttgcaaagcatccggctatacatttacggactactacatgaattggatgaggcagaggcacggcgagacccttgagtggataggagacatcaacccgaacaacggggacccgtcatacaatcagaagttcaaagataaagcaactcttactgttgataaatcctcaagcactgcgagcatggaactgaggagtcttacatccgacgattccgctgtttactattgcgccagggaaggtccttccttcgcttattggggtcaggggacattggttaccgtctccgca

SEQ ID NO: 40 - DNA, 396 bp, synthetic construct
  misc_feature 1..396: note = mAb KCD003 light chain
  source 1..396: mol_type = other DNA
atgtatcgaatgcagctcttgtcatgtatagccctttctctggctctcgttactaacagcgatgtgttgatgacacaaacccctctcagtctgcccgtttcacttggcgaacaggcgagtattagctgccgatcttcccaaactatagttcacagtaacggagatacgtacctggagtggtacctgcagaaaccgggccagtcacctaacttgctcatttacaaagtctcaaatagattctccggagttccagataggttttccggtagtggttctggtacggacttcactttgaagattagccgcgtcgaggcggaggaccttggggtctactattgcttccaaggctcccatgtgcctcccacgtttggaggaggcactaagcttgaaattaaa

SEQ ID NO: 41 - DNA, 408 bp, synthetic construct
  misc_feature 1..408: note = mAb KCD005 heavy chain
  source 1..408: mol_type = other DNA
atgtatcggatgcaactgttgagttgcattgcacttagtctcgcactcgtgacgaacagcgaggtacaactgcaacagtctggccctgaattggtaaaacccggtgcctctgttaaaatcagctgtaaagcgtccggctatacatttacagaccactatatgaattgggtcaagcaatctcatggaaaatcccttgaatggataggagatataaatccgaataacggaggcactagttgtaaccagaagtttaagggaaaagctactctgactgtggataaatcatcttccactgcctatatggagcttaggtctcttactagcggggactctgcggtctactactgcacccgcgaaggggcatctttcgctttctggggtcagggaacactggttacagtctctgca

SEQ ID NO: 42 - DNA, 396 bp, synthetic construct
  misc_feature 1..396: note = mAb KCD005 light chain
  source 1..396: mol_type = other DNA
atgtatagaatgcaattgttgtcatgtatcgcgctctcactcgcattggttactaactctgatgtgttgatgacgcagactcccctgtctctcccagtgtcacttggcgatcaggcttctatatcctgccgaagctcccagagcattgtccacagtaacggagacacttatctggaatggtacctccaaaagccgggccagagtccaaaacttttgatctataaagtcagtaatagatttagtggggttccagacaggttctcaggcagcggctcaggcaccgacttcactctgaagataagccgggttgaagctgaggatctcggggtgtattactgtttccaagggtcacatgtaccagttacatttggagcaggcactaagcttgaaattaaa

SEQ ID NO: 43 - protein, 116 aa, synthetic construct
  REGION 1..116: note = 34I54I59D84S heavy chain
EVQLVESGGGLVQPGGSLRLSCAASGYTFTDYYINWVRQAPGKGLEWIGDINPITGDTDYNADFKRRFTFSLDTSKSTAYLQMSSLRAEDTAVYYCTREGPSFAYWGQGTLVTVSS

SEQ ID NO: 44 - protein, 112 aa, synthetic construct
  REGION 1..112: note = 54R101V light chain
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPVTFGQGTKVEIK

SEQ ID NO: 45 - skipped

SEQ ID NO: 46 - protein, 8 aa, synthetic construct
  REGION 1..8: note = HCDR2
INPITGDT

SEQ ID NO: 47 - protein, 7 aa, synthetic construct
  REGION 1..7: note = HCDR3
EGPSFAY

SEQ ID NO: 48 - protein, 10 aa, synthetic construct
  REGION 1..10: note = LCDR1
QTIVHSNGDT

SEQ ID NO: 49 - skipped

SEQ ID NO: 50 - protein, 9 aa, synthetic construct
  REGION 1..9: note = LCDR3
FQGSHVPVT

SEQ ID NO: 51 - protein, 10 aa, synthetic construct
  REGION 1..10: note = Antigen binding protein components
GYDFTHYGMN

SEQ ID NO: 52 - protein, 17 aa, synthetic construct
  REGION 1..17: note = Antigen binding protein components
WINTYTGEPTYAADFKR

SEQ ID NO: 53 - protein, 14 aa, synthetic construct
  REGION 1..14: note = Antigen binding protein components
YPYYYGTSHWYFDV

SEQ ID NO: 54 - protein, 11 aa, synthetic construct
  REGION 1..11: note = Antigen binding protein components
SASQDISNYLN

SEQ ID NO: 55 - protein, 7 aa, synthetic construct
  REGION 1..7: note = Antigen binding protein components
FTSSLHS

SEQ ID NO: 56 - protein, 9 aa, synthetic construct
  REGION 1..9: note = Antigen binding protein components
QQYSTVPWT